(19) United States
(12) Patent Application Publication
(10) Pub. No.: US 2022/0401023 A1
(43) Pub. Date: Dec. 22, 2022

(54) NON-INVASIVE RADIOMIC SIGNATURE TO PREDICT RESPONSE TO SYSTEMIC TREATMENT IN SMALL CELL LUNG CANCER (SCLC)

(71) Applicants: Case Western Reserve University, Cleveland, OH (US); The United States Govemment as Represented by The Department of Veteran Affairs, Washington, DC (US)

(72) Inventors: Anant Madabhushi, Shaker Heights, OH (US); Mohammadhadi Khorrami, Cleveland Heights, OH (US); Prantesh Jain, Cleveland, OH (US); Afshin Dowlati, Solon, OH (US)

(21) Appl. No.: 17/530,711

(22) Filed: Nov. 19, 2021

Related U.S. Application Data
(60) Provisional application No. 63/212,247, filed on Jun. 18, 2021.

(51) Int. Cl. A61B 5/00; G06V 10/44; G06V 10/764; G06V 10/774; G06T 7/00; G16H 50/20; G16H 30/40; G16H 50/70; G16H 20/10; G16H 50/30

(52) U.S. Cl. CPC A61B 5/4848; A61B 5/7275; A61B 5/7267; G06V 10/44; G06V 10/764; G06V 10/774; G06T 7/0012; G16H 50/20; G16H 30/40; G16H 50/70; G16H 20/10; G16H 50/30; A61B 2576/02; G06T 2207/10116; G06T 2207/30096; G06T 2207/10081; G06T 2207/30061

(57) ABSTRACT

Various embodiments of the present disclosure are directed towards a method for predicting a response to treatment of small cell lung cancer (SCLC). The method includes generating a radiomic risk score (RRS) for the patient based on a plurality of radiomic features, wherein the RRS is prognostic of overall survival (OS) of the patient. The RRS is provided to a machine learning classifier that is trained to predict a response of the patient to a SCLC chemotherapy treatment based, at least in part, on the RRS. The machine learning classifier provides a classification of the patient into either a responder group (RG) or a non-responder group (NRG), where the NRG indicates the patient will not respond to the SCLC chemotherapy treatment and the RG indicates that the patient will respond to the SCLC chemotherapy treatment.

### REFERENCE TO RELATED APPLICATIONS

This application claims the benefit of U.S. Provisional Application No. 63/212,247 filed on Jun. 18, 2021, the contents of which are hereby incorporated by reference in their entirety.

### FEDERAL FUNDING NOTICE

This invention was made with government support under grants W81XWH-19-1-0668, W81XWH-15-1-0558, W81XWH-20-1-0851, W81XWH-18-1-0440, W81XWH-20-1-0595, and W81XWH-18-1-0404 awarded by the Department of Defense; grants CA199374, CA202752, CA208236, CA216579, CA220581, CA239055, CA248226, CA254566, HL151277, EB028736, RR012463, and TR000254 awarded by the National Institutes of Health; grant IBX004121A awarded by the United States Department of Veterans Affairs. The government has certain rights in the invention.

### BACKGROUND

Small cell lung cancer (SCLC) is a disease in which malignant (cancer) cells form in tissues of a lung. While SCLC accounts for only about 15% percent of lung cancers, SCLC is more aggressive than other types of lung cancer (e.g., SCLC cancer cells grow quickly and travel to other parts of the body more easily than other types of lung cancer). As a result, SCLC is usually diagnosed after the cancer has spread throughout the body (metastasized), making recovery less likely.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a method for generating a prediction for a response to a treatment for small cell lung cancer (SCLC).

FIG. 2 illustrates some embodiments of a method for generating a prediction for overall survival (OS) of a patient with SCLC.

FIG. 3 illustrates a method of some more detailed embodiments of the fourth operation of the method of FIG. 1.

FIG. 4 illustrates a method of some other embodiments for generating a prediction for a response to a treatment for SCLC.

FIG. 5 illustrates a method of some other embodiments of the method of FIG. 1.

FIG. 6 illustrates a method of some more detailed embodiments of the first operation of the method of FIG. 5.

FIGS. 7A-7C illustrate various views that are associated with Example Use Case 1.

FIG. 8 illustrates various plots that are associated with Example Use Case 1.

FIG. 9 illustrates some embodiments of an apparatus that can facilitate the methods described herein.

FIG. 10 illustrates some other embodiments of the apparatus of FIG. 9.

FIG. 11 illustrates some embodiments of a computer in which methods described herein can operate and in which example methods, apparatus, circuits, operations, or logics may be implemented.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Small Cell Lung Cancer (SCLC) is an aggressive malignancy that is characterized by high resistance to chemotherapy and rapid progression. Currently, there are no consistent predictive biomarkers that can accurately guide the use of systemic therapy in patients with SCLC.

Various embodiments of the present disclosure relates to a method (and related apparatus) to utilize quantitative radiomic features (e.g., computer extracted imaging) from scans (e.g., pre-treatment computed tomography (CT) scans) to predict a response and/or sensitivity to treatments (e.g., platinum-based chemotherapy) as well as prognosticate overall survival (OS). The method includes accessing a pre-treatment scan (e.g., CT scan) of a patient that is receiving or is to receive treatment for SCLC, where the pre-treatment scan demonstrates a pulmonary lesion that is indicative of SCLC. Thereafter, quantitative radiomic features are extracted from the pre-treatment scan from inside (intratumoral) and outside (peritumoral) the pulmonary lesion. A radiomic risk score (RRS), which is prognostic of OS of the patient, is generated for the patient based on a combination of the quantitative radiomic features. The RRS is then provided to a machine learning classifier that is trained to predict a response to a treatment (e.g., platinum-based SCLC chemotherapy) based, at least in part, on the RRS of the patient. By utilizing an RRS of a patient and a machine learning classifier to predict the response to a treatment, treatment of patients with SCLC may be accurately guided to achieve better treatment results (e.g., to expedite alternative treatment options, especially in non-responders).

Some portions of the detailed descriptions that follow are presented in terms of algorithms and symbolic representations of operations on data bits within a memory. These algorithmic descriptions and representations are used by those skilled in the art to convey the substance of their work to others. An algorithm, here and generally, is conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic or circuit, and so on. The physical manipulations create a concrete, tangible, useful, real-world result.

It has proven convenient at times, principally for reasons of common usage, to refer to these signals as bits, values, elements, symbols, characters, terms, numbers, and so on. It should be borne in mind, however, that these and similar terms are to be associated with the appropriate physical quantities and are merely convenient labels applied to these quantities. Unless specifically stated otherwise, it is appreciated that throughout the description, terms including processing, computing, calculating, determining, and so on, refer to actions and processes of a computer system, logic, circuit, processor, or similar electronic device that manipulates and transforms data represented as physical (electronic) quantities.

A processor(s) may include any combination of general-purpose processors and dedicated processors (e.g., graphics processors, application processors, etc.). The processors may be coupled with or may include memory or storage and may be configured to execute instructions stored in the memory or storage to enable various apparatus, applications, or operating systems to perform the operations or methods described herein. The memory or storage devices may include main memory, disk storage, or any suitable combination thereof. The memory or storage devices may include, but are not limited to any type of volatile or non-volatile memory such as dynamic random access memory (DRAM), static random-access memory (SRAM), erasable programmable read-only memory (EPROM), electrically erasable programmable read-only memory (EEPROM), Flash memory, or solid-state storage.

Example methods and operations may be better appreciated with reference to flow diagrams. While for purposes of simplicity of explanation, the illustrated methodologies are shown and described as a series of blocks, it is to be appreciated that the methodologies are not limited by the order of the blocks, as some blocks can occur in different orders and/or concurrently with other blocks from that shown and described. Moreover, less than all the illustrated blocks may be required to implement an example methodology. Blocks may be combined or separated into multiple components. Furthermore, additional and/or alternative methodologies can employ additional, not illustrated blocks.

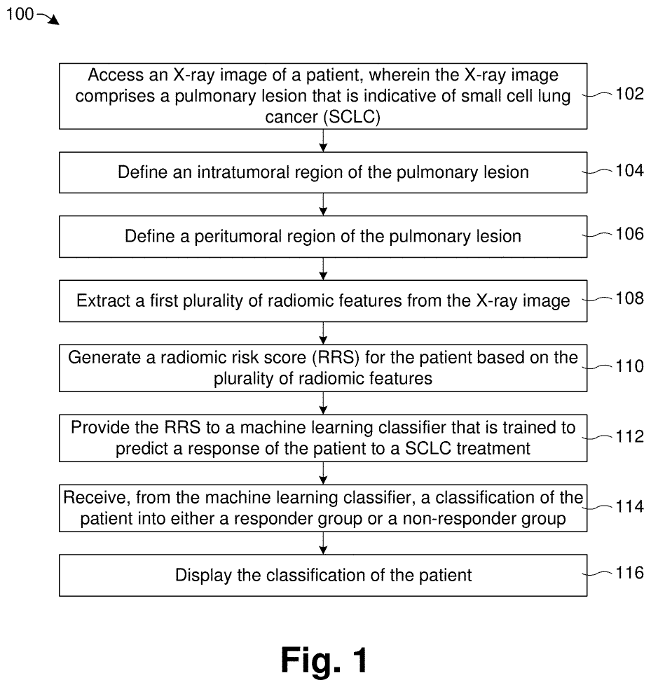

FIG. 1 illustrates some embodiments of a method 100 for generating a prediction for a response to a treatment for small cell lung cancer (SCLC). In some embodiments, the prediction gives a best objective response to chemotherapy based on the Response Evaluation Criteria in Solid Tumours (RECIST) criteria.

The method 100 comprises a first operation 102. At the first operation 102, an X-ray image of a patient that is receiving or is to receive treatment for SCLC is accessed. The X-ray image comprises a pulmonary lesion. The pulmonary lesion may be indicative of SCLC. In some embodiments, the X-ray image is from a computed tomography (CT) scan of the patient. In embodiments in which the X-ray image is from a CT scan of the patient, the X-ray image may be referred to as a CT image.

In some embodiments, the treatment for SCLC is a platinum-based chemotherapy treatment for SCLC (e.g., comprising the use of platinum-based drugs such as cisplatin, oxaliplatin, carboplatin, etc.). In further embodiments, the X-ray image is a CT image of the patient that was taken before the start of a platinum-based chemotherapy treatment for SCLC (e.g., a pre-treatment CT image).

The CT image may be stored in memory, either locally or remotely. The CT image may be obtained by a medical imaging device (e.g., a CT scanner). The CT image may be obtained concurrently with the method 100 (e.g., via the medical imaging device implementing method 100) or prior to the method 100 (e.g., at a time that is before a time in which the method 100 is implemented). Accessing the CT image includes acquiring electronic data, reading from a computer file, receiving a computer file, reading from a computer memory, or other computerized activity not practically performed in the human mind.

The method 100 comprises a second operation 104. At the second operation 104, an intratumoral region of the pulmonary lesion is defined. Defining the intratumoral region of the pulmonary lesion comprises defining an outer boundary of the pulmonary lesion. The area within the outer boundary of the pulmonary lesion is defined as the intratumoral region of the pulmonary lesion. Defining the intratumoral region of the pulmonary lesion includes acquiring electronic data, reading from a computer file, receiving a computer file, reading from a computer memory, or other computerized activity not practically performed in the human mind.

The method 100 comprises a third operation 106. At the third operation 106, a peritumoral region of the pulmonary lesion is defined. The peritumoral region of the pulmonary region is a region outside the outer boundary of the pulmonary lesion. In some embodiments, a process for defining the peritumoral region of the pulmonary lesion comprises enlarging the outer boundary of the pulmonary lesion to define an outer boundary of the peritumoral region. The area between the outer boundary of the pulmonary lesion and the outer boundary of the peritumoral region is defined as the peritumoral region of the pulmonary lesion. Defining the peritumoral region of the pulmonary lesion includes acquiring electronic data, reading from a computer file, receiving a computer file, reading from a computer memory, or other computerized activity not practically performed in the human mind.

In some embodiments, the outer boundary of the pulmonary lesion is enlarged by about 15 millimeters (mm) to generate the outer boundary of the peritumoral region. In other embodiments, the outer boundary of the pulmonary lesion may be enlarged by a predefined number of pixels/voxels (e.g., 3 pixels). As such, in some embodiments, the area of the peritumoral region may be expressed in millimeters (or some other unit), whereas in other embodiments the area of the peritumoral region may be expressed in pixels/voxels (or some other unit).

In some embodiments, a shape of the outer boundary of the pulmonary lesion is maintained during enlargement, such that the outer boundary of the peritumoral region has a same contour (e.g., shape) as the outer boundary of the pulmonary lesion. In further embodiments, a centroid of the boundary of the peritumoral region is aligned with a centroid of the outer boundary of the pulmonary lesion before the area between the outer boundary of the pulmonary lesion and the outer boundary of the peritumoral region is defined as the peritumoral region of the pulmonary lesion.

In some embodiments, the outer boundary of the pulmonary lesion may be defined by a radiologist. In further embodiments, the outer boundary of the peritumoral region, the intratumoral region, and/or the peritumoral region may be defined by the radiologist. In other embodiments, the outer boundary of the peritumoral region, the intratumoral region, and/or the peritumoral region may be defined using an image segmentation technique, such as, a watershed segmentation technique, a region growing technique, an active contour technique, a convolutional neural network (CNN), some other image segmentation technique, or a combination of the foregoing. It will be appreciated that the outer boundary of the peritumoral region and/or the outer boundary of the pulmonary lesion may be generated using other techniques.

The method 100 comprises a fourth operation 108. At the fourth operation 108, a first plurality of radiomic features are extracted from the X-ray image. The first plurality of radiomic features are extracted from the intratumoral region and/or the peritumoral region of the pulmonary lesion. The radiomic features of the first plurality of radiomic features are radiomic features that have been determined to be (e.g., via a feature selection process, such as least absolute shrinkage and selection operator (LASSO), minimum redundancy maximum relevance (mRMR), best subsets selection, correlation feature selection, etc.) more relevant (e.g., discriminative) radiomic features for predicting overall survival (OS) of patients with SCLC (e.g., the length of time from either the date of diagnosis or the start of treatment for SCLC that patients diagnosed with SCLC are still alive). Extracting the first plurality of radiomic features includes acquiring electronic data, reading from a computer file, receiving a computer file, reading from a computer memory, or other computerized activity not practically performed in the human mind.

In some embodiments, the first plurality of radiomic features comprises one or more shape-based features of the intratumoral region of the pulmonary lesion, one or more texture-based features of the intratumoral region of the pulmonary lesion, one or more shape-based features of the peritumoral region of the pulmonary lesion, and/or one or more texture-based features of the peritumoral region of the pulmonary lesion. In further embodiments, the first plurality of radiomic features comprises one or more Haralick features, one or more Laws features, and/or one or more Gabor features of the intratumoral region and/or one or more Haralick features, one or more Laws features, and/or one or more Gabor features of the peritumoral region. In yet further embodiments, the first plurality of radiomic features comprises a Haralick entropy feature of the intratumoral region, a Laws texture feature of the intratumoral region, a Laws texture feature of the peritumoral region, a low frequency Gabor feature of the intratumoral region, and a high frequency Gabor feature of the peritumoral region. The Laws texture feature of the intratumoral region and/or the Laws texture feature of the peritumoral region may detect patterns of heterogeneous enhancement and/or abnormal structure. In yet further embodiments, the first plurality of radiomic features consists of the Haralick entropy feature of the intratumoral region, the Laws texture feature of the intratumoral region, the Laws texture feature of the peritumoral region, the low frequency Gabor feature of the intratumoral region, and the high frequency Gabor feature of the peritumoral region. In such embodiments, the above 6 radiomic features are more relevant (e.g., discriminative) for predicting overall survival (OS) of patients with SCLC. It will be appreciated that, in other embodiments, the radiomic features may comprise other radiomic features or first order statistics associated with the members of the radiomic features.

The method 100 comprises a fifth operation 110. At the fifth operation 110, a radiomic risk score (RRS) is generated for the patient based on the first plurality of radiomic features. In some embodiments, generating the RRS comprises assigning a value to each of the radiomic features of the first plurality of radiomic features. In further embodiments, the values are based on the number of times a specific indicator (e.g., a difference in pixel intensity) occurs in the intratumoral region and/or peritumoral region. For example, in some embodiments, a first radiomic feature may be based on a difference in pixel intensities between the peritumoral region with a filter (e.g., low pass filter, high pass filter, etc.) and the peritumoral region without a filter.

In some embodiments, generating the RRS may include weighting the radiomic features based on corresponding coefficients (e.g., the values are multiplied by respective coefficients). In further embodiments, the coefficients are generated such that they maximize a regression analysis. For example, in some embodiments, the coefficients are generated such that they maximize a linear regression model between input and output. The coefficients may be generated by the feature selection model. For example, in some embodiments, the coefficients are generated by a LASSO technique (e.g., the weights are selected by a LASSO feature selection model).

The values (or weighted values) are then combined to generate the RRS for the patient. The values are combined based on a function. The function may comprise combining the values by, for example, addition, subtraction, multiplication, division, some other mathematical operator, or a combination of the foregoing. In some embodiments, the RRS is generated based on a linear combination of the values (or weighted values). The RRS is a number (e.g., a numerical value) that is based on the combination of the values (or weighted values). The RRS is prognostic of the OS of the patient (e.g., the RRS is predictive of OS of the patient). In some embodiments, the RRS is generated by using a LASSO technique. Generating the RRS for the patient includes acquiring electronic data, reading from a computer file, receiving a computer file, reading from a computer memory, or other computerized activity not practically performed in the human mind.

The method 100 comprises a sixth operation 112. At the sixth operation 112, the RRS is provided to a machine learning classifier. The machine learning classifier is trained to predict a response of the patient to a SCLC treatment. The machine learning classifier predicts the response of the patient to the SCLC treatment (e.g., SCLC chemotherapy treatment) based on, at least in part, the RRS. In some embodiments, the SCLC treatment is the platinum-based chemotherapy treatment for SCLC.

In some embodiments, the machine learning classifier is a linear discriminant analysis (LDA) classifier. In other embodiments, the machine learning classifier may be, for example, a quadratic discriminant analysis (QDA) classifier, a support vector machine (SVM) classifier, or some other machine learning classifier. Providing the RRS to the machine learning classifier includes acquiring electronic data, reading from a computer file, receiving a computer file, reading from a computer memory, or other computerized activity not practically performed in the human mind.

The method 100 comprises a seventh operation 114. At the seventh operation 114, a classification of the patient into either a responder group (RG) or a non-responder group (NRG) is received from the machine learning classifier. The RG indicates that the patient will respond to the SCLC chemotherapy treatment (e.g., platinum-based SCLC chemotherapy). The NRG indicates that the patient will not respond to the SCLC chemotherapy treatment.

The machine learning classifier classifies the patient into either the RG or the NRG based, at least in part, on the RRS (e.g., the machine classifier has been trained to predict a response to the SCLC treatment by classifying into either the RG or the NRG based, at least in part, on the RRS).

The machine learning classifier classifies the patient into either the RG or the NRG by generating a classification value (e.g., a numerical value) based, at least in part, on the RRS. For example, in some embodiments, if the classification value (e.g., the numerical value) is less than (or greater than) a threshold classification value, the machine learning classifier classifies the patient into the NRG. On the other hand, if the classification value is greater than or equal to (or less than or equal to) the threshold classification value, the machine classifier classifies the patient into the RG. In other embodiments, if the classification value is less than or equal to (or greater than or equal to) the threshold classification value, the machine learning classifier classifies the patient into the NRG; and if the classification value is greater than (or less than) the threshold classification value, the machine classifier classifies the patient into the RG. It will be appreciated that other classification techniques may be employed. In some embodiments, the classification is generated with an area under receiver operating characteristic curve (AUC) of at least about 0.7.

The machine learning classifier classifies the patient into either the RG or the NRG based on the RRS (e.g., the machine classifier has been trained to predict a response to the SCLC treatment by classifying into either the RG or the NRG based on the RRS). For example, the machine learning classifier generates the classification value based on the RRS of the patient.

In other embodiments, the machine learning classifier may classify the patient into either the RG or the NRG based on the RRS and at least one other feature that is prognostic of the OS of the patient (e.g., the machine classifier has been trained to predict a response to the SCLC treatment by classifying into either the RG or the NRG based on both the RRS and the at least one other feature). For example, the machine learning classifier generates the classification value based on a combination of the RRS of the patient and the at least one other feature that is prognostic of the OS of the patient. The at least one other feature that is prognostic of the OS of the patient is different than the RRS. In some embodiments, the at least one other feature that is prognostic of the OS of the patient is a stage of the patient's SCLC. In further embodiments, the stage of the patient's SCLC is either extensive stage or limited stage (e.g., one of two stages). The stage of the patient's SCLC may be determined by a medical practitioner (e.g., radiologist, chemotherapist, etc.) and/or by a processor configured to generate the patient's SCLC stage. The patient's SCLC stage may be generated before, after, or concurrently with generating the RRS of the patient.

The method 100 comprises an eighth operation 116. At the eighth operation 116, the classification is displayed. The classification may be displayed on, for example, a computer monitor, a smartphone display, a tablet display, or some other display device, or a combination of the foregoing. It will be appreciated that the classification may be displayed in other mediums (e.g., the classification may be printed on paper) in addition to, or in lieu of, displaying the classification on a display device.

In some embodiments, the classification may be displayed along with displaying one or more of the radiomic features, the RRS of the patient, the X-ray image, or a classification of the patient into an OS class (e.g., short-term or long-term). In some embodiments, displaying the classification also includes controlling a personalized medicine system, a computer monitor, or other display, to display operating parameters or characteristics of a machine learning classifier, during at least one of training and testing of the machine learning classifier, or during clinical operation of the machine learning classifier. In some embodiments, displaying the classification comprises selecting for the classification to be displayed via a graphical control element (e.g., by clicking/tapping on an item in a drop-down list). Displaying the classification includes acquiring electronic data, reading from a computer file, receiving a computer file, reading from a computer memory, or other computerized activity not practically performed in the human mind.

By displaying the classification, a medical practitioner may be able easily (e.g., intuitively due to the single classification being displayed) and timely (e.g., due to the classification being based on pre-treatment CT images) predict the patient's response to the SCLC treatment (e.g., platinum-based SCLC chemotherapy).

Accordingly, the medical practitioner may be able to accurately guide the SCLC treatment of the patient to achieve better treatment results (e.g., to expedite alternative treatment options (e.g., adjuvant therapy, active surveillance, etc.) for the patient, if needed (e.g., the patient is in the NRG)).

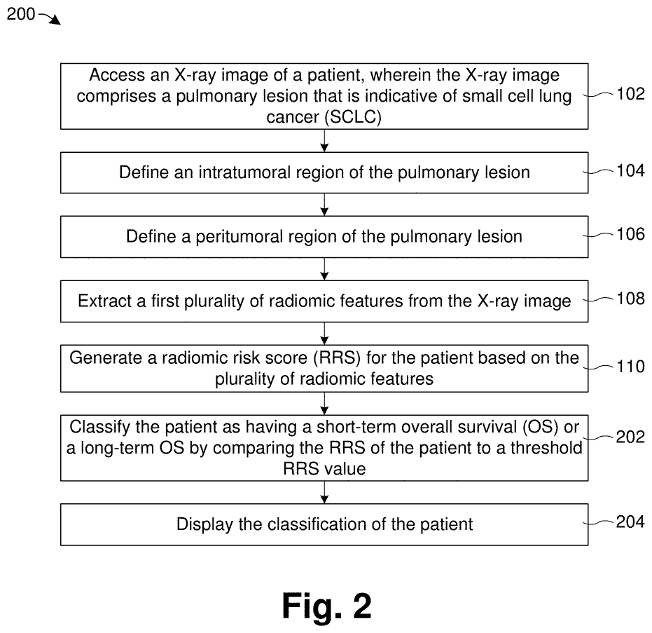

FIG. 2 illustrates some embodiments of a method 200 for generating a prediction for overall survival (OS) of a patient with SCLC. The method 200 comprises operations 102-110 as described herein.

The method 200 comprises a first operation 202. At the first operation 202, a patient is classified into either a short-term overall survival (OS) group or a long-term OS group by comparing the radiomic risk score (RRS) of the patient to a threshold RRS value. For example, if the RRS (e.g., the numerical value) of the patient is less than (or greater than) the threshold RRS value, the patient is classified into the short-term OS group. On the other hand, if the RRS of the patient is greater than or equal to (or less than or equal to) the threshold RRS value, the patient is classified into the long-term OS group. In some embodiments, the threshold RRS value is the median threshold RRS value of a group of patients (e.g., a training dataset). It will be appreciated that other comparisons of the RRS to the threshold RRS value may be utilized to classify the patient into either the short-term OS group or the long-term OS group. For example, if the RRS of the patient is less than or equal to (or greater than or equal to) the threshold RRS value, the patient is classified into the short-term OS group. On the other hand, if the RRS of the patient is greater than (or less than) the threshold RRS value, the patient is classified into the long-term OS group. In some embodiments, the threshold RRS value may be, for example, 0, 0.5, 1, or some other numerical value.

The short-term OS group indicates that the patient is likely to die before a threshold date. The long-term OS group indicates that the patient is likely to die after (or on) the threshold date. The patient may be classified into either the short-term OS group or the long-term OS group by comparing the RRS of the patient to a threshold RRS value due to a statistical model indicating that RRS is significantly assocaited with OS (e.g., a Cox regression analysis produced a statistically significant result that indicated the RRS of a patient corresponds to the OS of the patient). The threshold date is a predefined time (e.g., days, months, etc.) from either the date of diagnosis or the start of treatment for SCLC that patients diagnosed with SCLC are still alive. For example, in some embodiments, the threshold date may be about 9 months (e.g., 9.37 months), which is a median OS for a group of patients that have SCLC.

The method 200 comprises a second operation 204. At the second operation 204, the classification is displayed. The classification may be displayed on, for example, a computer monitor, a smartphone display, a tablet display, or some other display device, or a combination of the foregoing. It will be appreciated that the classification may be displayed in other mediums (e.g., the classification may be printed on paper) in addition to, or in lieu of, displaying the classification on a display device.

In some embodiments, the classification may be displayed along with displaying one or more of the radiomic features, the RRS of the patient, the X-ray image, or a classification of the patient into a treatment responder/non-responder group (e.g., the RG or the NRG). In some embodiments, displaying the classification also includes controlling a personalized medicine system, a computer monitor, or other display, to display operating parameters or characteristics of a machine learning classifier, during at least one of training and testing of the machine learning classifier, or during clinical operation of the machine learning classifier. In some embodiments, displaying the classification comprises selecting for the classification to be displayed via a graphical control element (e.g., by clicking/tapping on an item in a drop-down list). Displaying the classification includes acquiring electronic data, reading from a computer file, receiving a computer file, reading from a computer memory, or other computerized activity not practically performed in the human mind.

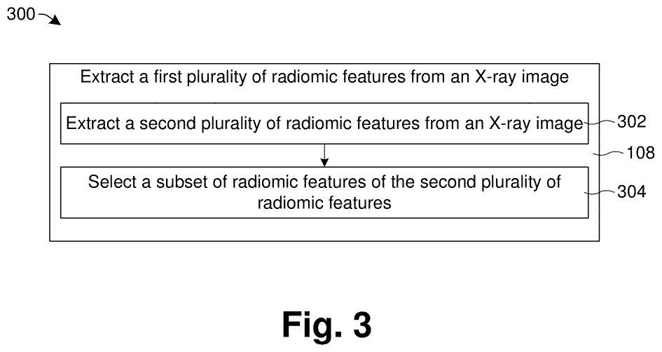

FIG. 3 illustrates a method 300 of some more detailed embodiments of the fourth operation 108 of the method 100 of FIG. 1. In other words, the method 300 illustrates some more detailed embodiments of extracting a first plurality of radiomic features from an X-ray image of a patient.

As shown in the method 300, in some embodiments, extracting the first plurality of radiomic features from the X-ray image of the patient comprises a first operation 302. At the first operation 302, a second plurality of radiomic features are extracted from the X-ray image. The radiomic features of the second plurality of radiomic features are extracted from the intratumoral region and/or the peritumoral region of the pulmonary lesion. Extracting the second plurality of radiomic features includes acquiring electronic data, reading from a computer file, receiving a computer file, reading from a computer memory, or other computerized activity not practically performed in the human mind.

In some embodiments, the second plurality of radiomic features comprises one or more shape-based features of the intratumoral region of the pulmonary lesion, one or more texture-based features of the intratumoral region of the pulmonary lesion, one or more shape-based features of the peritumoral region of the pulmonary lesion, and/or one or more texture-based features of the peritumoral region of the pulmonary lesion. In further embodiments, the second plurality of radiomic features comprises one or more Haralick features, one or more Laws features, and/or one or more Gabor features of the intratumoral region and/or one or more Haralick features, one or more Laws features, and/or one or more Gabor features of the peritumoral region. In yet further embodiments, the second plurality of radiomic features comprises a Haralick entropy feature of the intratumoral region, a Laws texture feature of the intratumoral region, a Laws texture feature of the peritumoral region, a low frequency Gabor feature of the intratumoral region, and a high frequency Gabor feature of the peritumoral region. The Laws texture feature of the intratumoral region and/or the Laws texture feature of the peritumoral region may detect patterns of heterogeneous enhancement and/or abnormal structure.

The method 300 further comprises a second operation 304. At the second operation 304, a subset of radiomic features of the second plurality of radiomic features are selected. The subset of radiomic features define the first plurality of radiomic features. In other words, the first plurality of radiomic features consist of the subset of radiomic features of the second plurality of radiomic features.

The subset of radiomic features are selected from the second plurality of radiomic features by determining which radiomic features of the second plurality of radiomic features are more relevant (e.g., the most discriminative) for predicting overall survival (OS) of patients with SCLC. A feature selection process determines which radiomic features of the second plurality of radiomic features are more relevant (e.g., discriminative) for predicting OS of patients with SCLC. The radiomic features of the second plurality of radiomic features that are found (e.g., via the feature selection process) to be more relevant (e.g., the most discriminative) for predicting OS of patients with SCLC are selected as the subset of radiomic features.

In some embodiments, the feature selection process may be, for example, LASSO, mRMR, best subsets selection, correlation feature selection, or the like. In further embodiments, the feature selection is LASSO. In embodiments in which the feature selection is LASSO, the subset of radiomic features may be more relevant than another, different subset of radiomic features of the second plurality of radiomic features which were selected by a different feature selection process (e.g., mMRM). In some embodiments, the first plurality of radiomic features is referred to as a first set of radiomic features, and the second plurality of radiomic features is referred to as a second set of radiomic features. In further embodiments, selecting the subset of radiomic features includes acquiring electronic data, reading from a computer file, receiving a computer file, reading from a computer memory, or other computerized activity not practically performed in the human mind.

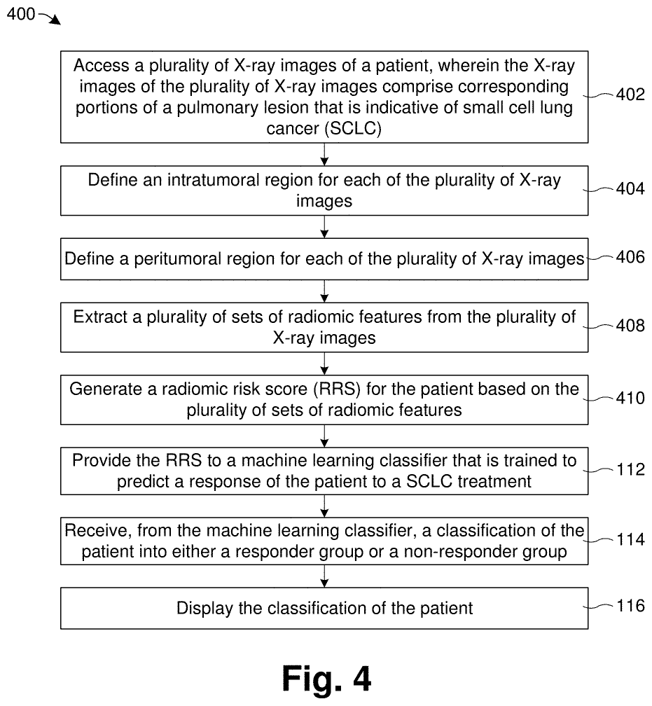

FIG. 4 illustrates a method 400 of some other embodiments for generating a prediction for a response to a treatment for SCLC. The method 400 comprises operations 112-116 as described herein. In some embodiments, the prediction gives a best objective response to chemotherapy based on the RECIST criteria.

The method 400 comprises a first operation 402. At the first operation 402, a plurality of X-ray images of a patient that is receiving or is to receive treatment for SCLC are accessed. The plurality of X-ray image comprise corresponding portions of a pulmonary lesion. For example, assume the plurality of X-ray images comprises 10 X-ray images. In such embodiments, there are 10 corresponding portions (e.g., 10 different slices) of the pulmonary lesion, and each of the 10 X-ray images comprises one of the 10 corresponding portions of the pulmonary lesion. The pulmonary lesion may be indicative of SCLC. Each of the plurality of X-ray images is a slice (e.g., cross-sectional area along a plane) of the patient's pulmonary lesion. In some embodiments, the plurality of X-ray images are from a computed tomography (CT) scan of the patient. The first operation 402 is substantially similar to the first operation 102, except the first operation 402 accesses a plurality of X-ray images whereas the first operation 102 accesses an X-ray image. It will be appreciated that, in some embodiments, the X-ray image of the method 100 is one of the X-ray images of the plurality of X-ray images (e.g., a first X-ray image of the plurality of X-ray images). In some embodiments, the corresponding portions of the pulmonary lesion may be more generally referred to as pulmonary lesions.

The method 400 comprises a second operation 404. At the second operation 404, an intratumoral region is defined for each of the plurality of X-ray images. For example, an intratumoral region is defined for each of the 10 X-ray images, such that there are 10 different intratumoral regions. The intratumoral regions may vary (e.g., in size and shape) slightly from one another (e.g., due to the 3D shape of the pulmonary lesion). The second operation 404 is substantially similar to the second operation 104, except the second operation 404 defines an intratumoral region for each of the plurality of X-ray images whereas the second operation 104 defines an intratumoral region for an X-ray image. It will be appreciated that, in some embodiments, the intratumoral region of the pulmonary lesion of the method 100 is one of the intratumoral regions of the plurality of X-ray images.

The method 400 comprises a third operation 406. At the third operation 406, a peritumoral region is defined for each of the plurality of X-ray images. For example, a peritumoral region is defined for each of the 10 X-ray images, such that there are 10 different peritumoral regions. The peritumoral regions may vary (e.g., in size and shape) slightly from one another (e.g., due to the 3D shape of the pulmonary lesion). The third operation 406 is substantially similar to the third operation 106, except the third operation 406 defines a peritumoral region for each of the plurality of X-ray images whereas the third operation 106 defines a peritumoral region for an X-ray image. It will be appreciated that, in some embodiments, the peritumoral region of the pulmonary lesion of the method 100 is one of the peritumoral regions of the plurality of X-ray images.

The method 400 comprises a fourth operation 408. At the fourth operation 408, a plurality of sets of radiomic features are extracted from the plurality of X-ray images. For example, the plurality of sets of radiomic features comprises a first set of radiomic features that are extracted from a first X-ray image of the 10 X-ray images, the plurality of sets of radiomic features comprises a second set of radiomic features that are extracted from a second X-ray image of the 10 X-ray images, and so forth. The radiomic features of the sets of radiomic features are extracted from the intratumoral region and/or the peritumoral region of their corresponding X-ray image. For example, the radiomic features of the first set of radiomic features are extracted from the intratumoral region and/or the peritumoral region of the first X-ray image. The fourth operation 408 is substantially similar to the fourth operation 108, except the fourth operation 408 extracts a set of radiomic features from each of the plurality of X-ray images whereas the fourth operation 108 extracts a plurality (e.g., a set) of radiomic features from an X-ray image. It will be appreciated that, in some embodiments, the first plurality of radiomic features of the method 100 is one of the sets of radiomic features of the plurality of sets of radiomic features.

The method 400 comprises a fifth operation 410. At the fifth operation 410, a radiomic risk score (RRS) is generated for the patient based on the plurality of sets of radiomic features. In some embodiments, generating the RRS comprises assigning a value to each of the radiomic features of the sets of radiomic features. In further embodiments, the values are based on the number of times a specific indicator (e.g., a difference in pixel intensity) occurs in the intratumoral region and/or peritumoral region of a corresponding X-ray image.

For example, in some embodiments, a first radiomic feature of the first set of radiomic features may be based on a difference in pixel intensities between the peritumoral region of the first X-ray image with a first filter (e.g., low pass filter) and the peritumoral region of the first X-ray image without the first filter. A second radiomic feature of the first set of radiomic features may be based on a difference in pixel intensities between the peritumoral region of the first X-ray image with a second filter (e.g., high pass filter) and the peritumoral region of the first X-ray image without the second filter. A first radiomic of the second set of radiomic features may be based on a difference in pixel intensities between the peritumoral region of the second X-ray image with the first filter (e.g., low pass filter) and the peritumoral region of the second X-ray image without the first filter. A second radiomic feature of the second set of radiomic features may be based on a difference in pixel intensities between the peritumoral region of the second X-ray image with the second filter (e.g., high pass filter) and the peritumoral region of the second X-ray image without the second filter.

In some embodiments, generating the RRS for the patient based on the plurality of sets of radiomic features further comprises categorizing the radiomic features of each set of radiomic features into radiomic feature types. The radiomic feature types correspond to the specific indicator type of the radiomic features. For example, the first radiomic feature of the first set of radiomic features and the first radiomic feature of the second set of radiomic features are classified into a first radiomic feature type (e.g., due to these radiomic features both being assigned a value in the same way (e.g., use of the first filter)), and the second radiomic feature of the first set of radiomic features and the second radiomic feature of the second set of radiomic features are classified into a second radiomic feature type (e.g., due to these radiomic features both being assigned a value in the same way (e.g., use of the second filter)).

The values of the radiomic features of the sets of radiomic features that have the same radiomic feature type are combined to generate a plurality of combined values. Each of the plurality of combined values corresponds to one of the radiomic feature types. For example, assume the first radiomic feature of the first set of radiomic features was assigned a first value (e.g., 100, due to 100 specific indicators occurring in the first X-ray image with and without the first filter), the first radiomic feature of the second set of radiomic features was assigned a second value (e.g., 50), the second radiomic feature of the first set of radiomic features was assigned a third value (e.g., 50), and the second radiomic feature of the second set of radiomic features was assigned a fourth value (e.g., 10). The first value and the second value would be combined (e.g., a mean, a median, some other statistical combination, etc.) to generate a first combined value (e.g., 75), and the third value and the fourth value would be combined (e.g., a mean, a median, some other statistical combination, etc.) to generate a second combined value (e.g., 30).

In some embodiments, generating the RRS may include weighting the plurality of combined values based on corresponding coefficients (e.g., the values are multiplied by respective coefficients). In further embodiments, the coefficients are generated such that they maximize a regression analysis. For example, in some embodiments, the coefficients are generated such that they maximize a linear regression model between input and output. The coefficients may be generated by the feature selection model. For example, in some embodiments, the coefficients are generated by a LASSO technique (e.g., the weights are selected by a LASSO feature selection model).

The RRS for the patient is then generated based on the plurality of combined values (or the plurality of weighted combined values). The RRS is generated based on the plurality of combined values by combining the plurality of combined values based on a function. The function may comprise combining the values by, for example, addition, subtraction, multiplication, division, some other mathematical operator, or a combination of the foregoing. In some embodiments, the RRS is generated based on a linear combination of the values (or weighted values). The RRS is a number (e.g., a numerical value) that is based on the combination of the combined values (or weighted combined values). The RRS is prognostic of the OS of the patient (e.g., the RRS is predictive of OS of the patient). In some embodiments, the RRS is generated by using a LASSO technique. Because the first plurality of radiomic features of the method 100 may be one of the sets of radiomic features of the plurality of sets of radiomic features, it will be appreciated that generating the RRS for the patient based on the plurality of combined values also includes generating the RRS for the patient based on the first plurality of radiomic features. The fifth operation 410 is substantially similar to the fifth operation 110, except the fifth operation 410 generates an RRS based on the plurality of combined values (or the plurality of weighted combined values) of the plurality of sets of radiomic features whereas the fifth operation 110 generates an RRS based on the values (or weighted values) of the radiomic features of the first plurality of radiomic features.

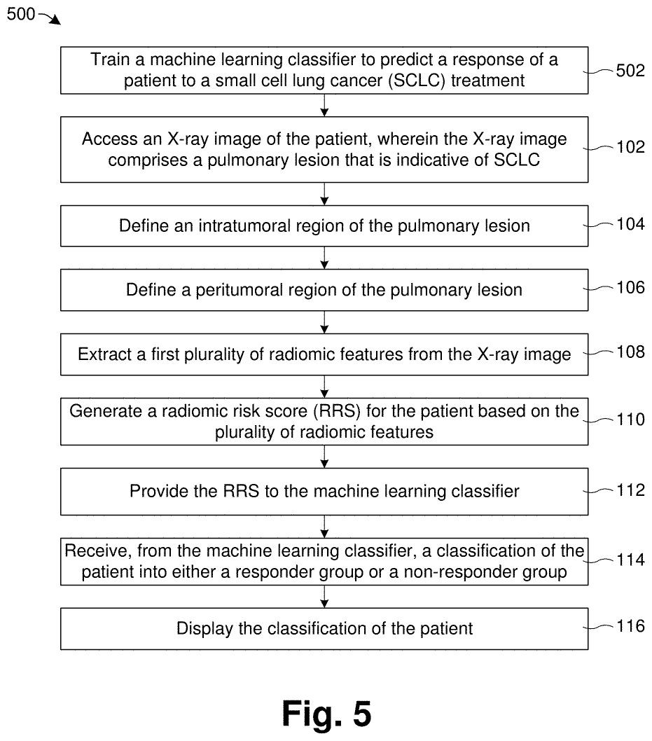

FIG. 5 illustrates a method 500 of some other embodiments of the method of FIG. 1. More specifically, the method 500 is similar to the method 100 of FIG. 1 and includes operations 102-116, but also includes a first operation 502. At the first operation 502, a machine learning classifier is trained to predict a response of a patient to a small cell lung cancer (SCLC) treatment (e.g., platinum-based SCLC chemotherapy).

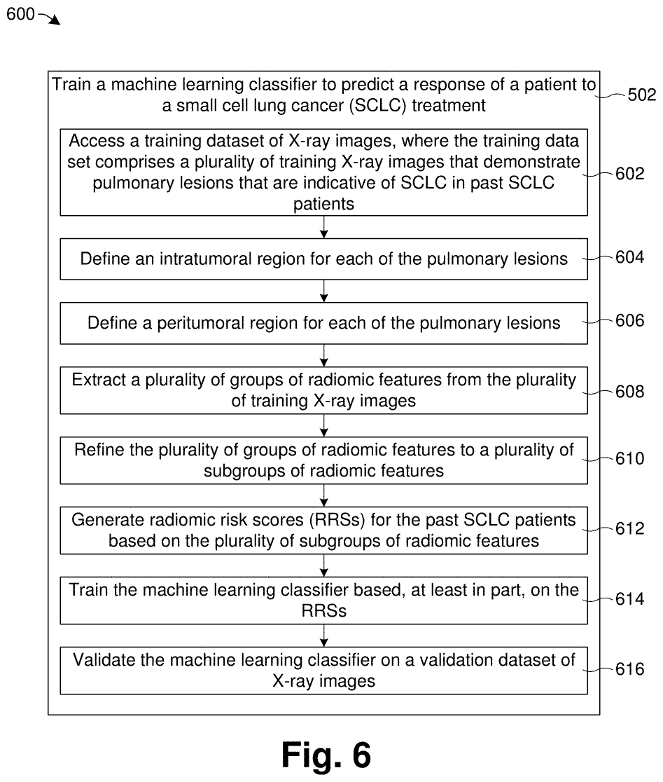

FIG. 6 illustrates a method 600 of some more detailed embodiments of the first operation 502 of the method 500 of FIG. 5. In other words, the method 600 illustrates some more detailed embodiments of training a machine learning classifier to predict a response of a patient to a SCLC treatment.

The method 600 comprises a first operation 602. At the first operation 602, a training dataset of X-ray images is accessed. The training dataset comprises a plurality of training X-ray images (e.g., data from the plurality of training X-ray images). Each of the plurality of training X-ray images comprises a pulmonary lesion (or region of a pulmonary lesion) that is indicative of SCLC. Further, each of the plurality of training X-ray images is associated with a past SCLC patient.

For example, the plurality of training X-ray images comprises a first X-ray training image, a second X-ray training image, and so forth. The first training image is associated with a first past SCLC patient (e.g., a first human who was diagnosed with SCLC at an earlier time), the second training image is associated with a second past SCLC patient (e.g., a second (different) human who was diagnosed with SCLC at an earlier time), and so forth. In some embodiments, the plurality of training X-ray images are CT images of the past SCLC patients. Accessing the training dataset includes acquiring electronic data, reading from a computer file, receiving a computer file, reading from a computer memory, or other computerized activity not practically performed in the human mind.

The method 600 comprises a second operation 604. At the second operation 604, an intratumoral region is defined for each of the pulmonary lesions. For example, the first training image comprises a first pulmonary lesion, and the second training image comprises a second pulmonary lesion. A first intratumoral region is defined for the first pulmonary lesion, and the second intratumoral region is defined for the second pulmonary lesion. The second operation 604 is substantially similar to the second operation 104, except the second operation 604 defines an intratumoral region for each of the pulmonary lesions (each of the plurality of training X-ray images) whereas the second operation 104 defines an intratumoral region for a pulmonary lesion (an X-ray image).

The method comprises 600 a third operation 606. At the third operation 606, a peritumoral region is defined for each of the pulmonary lesions. For example, a first peritumoral region is defined for the first pulmonary lesion, and a second peritumoral region is defined for the second pulmonary lesion. The third operation 606 is substantially similar to the third operation 106, except the third operation 606 defines a peritumoral region for each of the pulmonary lesions (each of the plurality of training X-ray images) whereas the third operation 106 defines a peritumoral region for a pulmonary lesion (an X-ray image).

The method 600 comprises a fourth operation 608. At the fourth operation 608, a plurality of groups of radiomic features are extracted from the plurality of training X-ray images. Each of the plurality of groups of radiomic features is associated with a corresponding training X-ray image of the plurality of training X-ray images. For example, a first group of radiomic features of the plurality of groups of radiomic features are extracted from (and thus associated with) the first training image, and a second group of radiomic features of the plurality of groups of radiomic features are extracted from the second training image.

For each group of radiomic features, each radiomic feature relates to an attribute (e.g., a difference in pixel intensity between the use of and non-use of a filter) of a corresponding intratumoral region and/or an attribute of a corresponding peritumoral region. In other words, the radiomic features of the groups of radiomic features are extracted from (and thus related to) the intratumoral region and/or the peritumoral region of their corresponding X-ray image. For example, the radiomic features of the first group of radiomic features are extracted from (and thus related to) the intratumoral region and/or the peritumoral region of the first training image, and the radiomic features of the second group of radiomic features are extracted from the intratumoral region and/or the peritumoral region of the second training image. The fourth operation 608 is substantially similar to the fourth operation 108, except the fourth operation 608 extracts a group of radiomic features from each of the training X-ray images whereas the fourth operation 108 extracts a plurality (e.g., a group) of radiomic features from an X-ray image.

The method 600 comprises a fifth operation 610. At the fifth operation 610, the plurality of groups of radiomic features are refined to a plurality of subgroups of radiomic features. For example, the first group of radiomic features is refined to a first subgroup of radiomic features, and the second group of radiomic features is refined to a second subgroup of radiomic features. As such, each subgroup of the plurality of subgroups of radiomic features is associated with a corresponding X-ray training image. For example, the first subgroup of radiomic features is associated with the first training image (e.g., due to the first subgroup of radiomic features comprising a smaller group of radiomic features of the first group of radiomic features).

The radiomic features of the subgroups of radiomic features are more relevant (e.g., more discriminative) to predicting overall survival (OS) of the past SCLC patients than the other radiomic features of the plurality of groups of radiomic features. A feature selection process determines which radiomic features of the plurality of groups of radiomic features are more relevant to OS of the past SCLC patients than the other radiomic features of the plurality of groups of radiomic features. The plurality of groups of radiomic features are then refined to the plurality of subgroups of radiomic features, such that the plurality of subgroups of radiomic features comprises the radiomic features that are more relevant to OS of the past SCLC patients.

In some embodiments, the feature selection process may be, for example, LASSO, mRMR, best subsets selection, correlation feature selection, or the like. In further embodiments, the feature selection is LASSO. In embodiments in which the feature selection is LASSO, the subgroups of radiomic features may be more relevant to OS of the past patients than other, different subgroups of radiomic features of the plurality of groups of radiomic features which were selected by a different feature selection process (e.g., mMRM). In further embodiments, refining the plurality of groups of radiomic features includes acquiring electronic data, reading from a computer file, receiving a computer file, reading from a computer memory, or other computerized activity not practically performed in the human mind.

The method 600 comprises a sixth operation 612. At the sixth operation 612, radiomic risk scores (RRSs) are generated for the past SCLC patients, respectively. For example, a first RRS is generated for the first past SCLC patient, and a second RRS is generated for the second past SCLC patient. Each of the RRSs is generated based on a corresponding subgroup of radiomic features. For example, a first RRS is generated based on the first subgroup of radiomic features, and a second RRS is generated based on the second subgroup of radiomic features. Each of the RRSs is prognostic of OS of a corresponding past SCLC patient. For example, the first RRS is prognostic of the OS of the first past SCLC patient, and the second RRS is prognostic of the OS of the second past SCLC patient. The sixth operation 612 is substantially similar to the fifth operation 110, except the sixth operation 612 generates an RRS for each of the past SCLC patients whereas the fifth operation 110 generates an RRS for a patient that is receiving or is to receive treatment for SCLC. It will be appreciated that the operations of the method 400 may be utilized in the method 600 (e.g., to generate each of the RRSs based on a corresponding plurality of set of radiomic features).

The method 600 comprises a seventh operation 614. At the seventh operation 614, the machine learning classifier is trained based, at least in part, on the RRSs. The machine learning classifier is trained to predict a response to a SCLC treatment (e.g., platinum-based SCLC chemotherapy) for a new SCLC patient (e.g., a new patient that is receiving or is to receive treatment for SCLC). In some embodiments, the machine learning classifier is a linear discriminant analysis (LDA) classifier. In other embodiments, the machine learning classifier may be, for example, a quadratic discriminant analysis (QDA) classifier, a support vector machine (SVM) classifier, or some other machine learning classifier. In further embodiments, the machine learning classifier may be trained based on, at least in part, the RRSs and at least one other feature that is prognostic of the OS (e.g., stage of past patients SCLC). Training the machine learning classifier includes acquiring electronic data, reading from a computer file, receiving a computer file, reading from a computer memory, or other computerized activity not practically performed in the human mind.

The method 600 comprises an eighth operation 616. At the eighth operation 616, the machine learning classifier is validated on a validation dataset of X-ray images. The validation dataset comprises a plurality of validation X-ray images. Each of the plurality of validation X-ray images comprises a pulmonary lesion (or region of a pulmonary lesion) that is indicative of SCLC. Further, each of the plurality of validation X-ray images is associated with a past SCLC patient.

Further, the validation dataset and the training dataset are portions of an original dataset (e.g., a larger collection of X-ray images having SCLC pulmonary lesions). The original dataset comprises a plurality of original X-ray images. Each of the plurality of original X-ray images comprises a pulmonary lesion (or region of a pulmonary lesion) that is indicative of SCLC. Further, each of the plurality of original X-ray images is associated with a past SCLC patient.

The original dataset is partitioned into the validation dataset and the training dataset. In some embodiments, the original dataset is partitioned into the validation dataset and the training dataset by randomly placing the original X-ray images into either the validation dataset or the training dataset. The machine learning classifier is validated on the validation dataset to ensure that the machine learning classifier is adequately able to predict a response to a SCLC treatment for a new SCLC patient (e.g., to determine the machine learning classifier is robust). In some embodiments, a k-fold cross-validation is utilized to validate the machine learning classifier. It will be appreciated that, in other embodiments, other cross-validation techniques may be utilized to validate the machine learning classifier.

### Example Use Case 1

An example embodiment includes training a machine learning classifier (e.g., prognostic classifier) to predict response to a SCLC treatment (e.g., platinum-based chemotherapy).

### Methods

In this example, 180 patients (e.g., past SCLC patients) with limited or extensive stage SCLC that received platinum-based chemotherapy were selected for this study. Of these, 27 patients were excluded for whom there was no measurable disease or the response to treatment was not available. The remaining 153 patients were randomly divided into a training set (n=77) and a validation set (n=76). The training set is one example of the training dataset of method 600. The validation set is one example of the validation dataset of method 600.

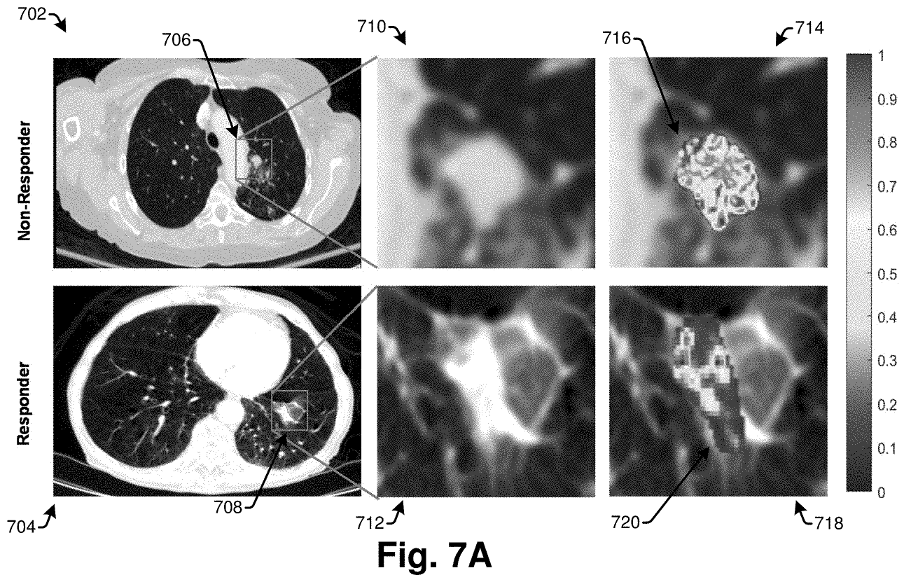

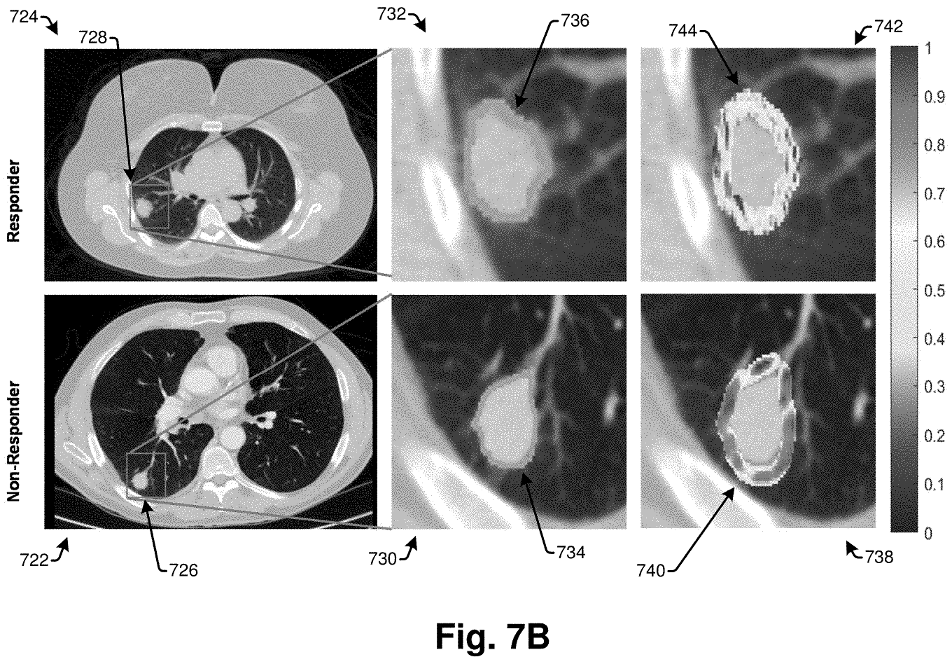

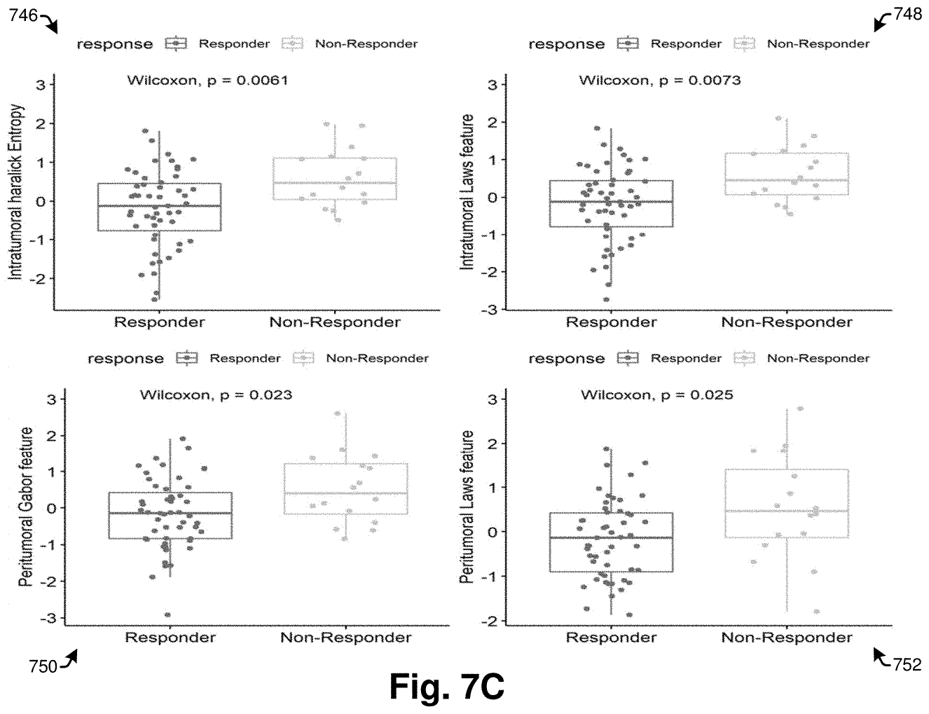

Lung tumors were contoured on 3D-Slicer® software by an expert reader (e.g., a radiologist). 1542 radiomic features (textural and shape) were extracted from intratumoral and peritumoral regions (15 mm region around the tumor). FIGS. 7A-7C illustrate various views that are associated with Example Use Case 1.

More specifically, FIG. 7A illustrates segmented tumor regions and heatmaps of intratumoral Haralick (entropy) feature in pre-treatment CT scans for representative non-responder and responder patients. More specifically, FIG. 7A illustrates a first pre-treatment CT image 702 of a representative non-responder patient and a second pre-treatment CT image 704 of a representative responder patient. The first pre-treatment CT image 702 has a first tumor region 706. The second pre-treatment CT image 704 has a second tumor region 708. Further, FIG. 7A illustrates a magnified view 710 of the first tumor region 706 and a magnified view 712 of the second tumor region 708. As seen in the magnified view 710, the first tumor region 706 has (e.g., illustrates) a first pulmonary lesion. As seen in the magnified view 712, the second tumor region 708 has (e.g., illustrates) a second pulmonary lesion. Moreover, FIG. 7A illustrates a first heatmap 714 of the intratumoral Haralick (entropy) feature in an intratumoral region 716 of the first pulmonary lesion. Also, FIG. 7A illustrates a second heatmap 718 of the intratumoral Haralick (entropy) feature in an intratumoral region 720 of the second pulmonary lesion. As seen in FIG. 7A, there is a difference in expression (e.g., an overexpression) of the intratumoral Haralick (entropy) features in the non-responder (e.g., the first tumor region 706) compared to the responder (e.g., the second tumor region 708). The first and second pre-treatment CT scans are examples of the pre-treatment X-ray images of the past SCLC patients described in method 600. The intratumoral regions illustrated in FIG. 7A are some examples of the intratumoral region(s) described in methods 100-600.

In addition, FIG. 7B illustrates segmented tumor regions and heatmaps of peritumoral Gabor feature in pre-treatment CT scans for representative non-responder and responder patients. More specifically, FIG. 7B illustrates a third pre-treatment CT image 722 of a representative non-responder patient and a fourth pre-treatment CT image 724 of a representative responder patient. The third pre-treatment CT image 722 has a third tumor region 726. The fourth pre-treatment CT image 724 has a fourth tumor region 728. Further, FIG. 7B illustrates a magnified view 730 of the third tumor region 726 and a magnified view 732 of the fourth tumor region 728. As seen in the magnified view 730, the third tumor region 726 has a third pulmonary lesion. As seen in the magnified view 732, the fourth tumor region 728 has a fourth pulmonary lesion. Also shown in the magnified view 730, the third pulmonary lesion has an outer boundary 734. Also shown in the magnified view 732, the fourth pulmonary lesion has an outer boundary 736. Moreover, FIG. 7B illustrates a third heatmap 738 of the peritumoral Gabor feature in a peritumoral region 740 of the third pulmonary lesion. Also, FIG. 7B illustrates a fourth heatmap 742 of the peritumoral Gabor feature in a peritumoral region 744 of the fourth pulmonary lesion. As seen in FIG. 7B, there is a difference in expression of the peritumoral Gabor features in the non-responder (e.g., the third tumor region 726) compared to the responder (e.g., the fourth tumor region 728). The third and fourth pre-treatment CT scans are examples of the pre-treatment X-ray images of the past SCLC patients described in method 600. The peritumoral regions and the outer boundaries illustrated in FIG. 7B are some examples of the peritumoral region(s) and outer boundary/boundaries described in methods 100-600.

In addition, FIG. 7C illustrates a collection of box and whisker plots 746-752. The collection of box and whisker plots illustrate radiomic features (e.g., 4 radiomic features) that best distinguish response to the SCLC treatment.

The primary endpoints of this study were overall survival and best objective response to chemotherapy defined per response evaluation criteria in solid tumors (RECIST) criteria. Patients with complete response were defined as “responders” (R) and those with stable or progression of disease were defined as “non-responders” (NR). A radiomic risk score (RRS) was generated by using least absolute shrinkage and selection operator (LASSO) and Cox regression model was used to predict overall survival (OS) in a univariate and multivariate setting. Kaplan—Meier survival analysis and log-rank statistical tests were performed to assess the discriminative ability of the radiomic features.

Those features that were prognostic of OS (e.g., RRS) were used to train a machine learning classifier to predict response to chemotherapy. A linear discriminant analysis (LDA) classifier was trained and then used to predict response to chemotherapy in SCLC patients. The area under the receiver operating characteristic curve (AUC) was calculated for objective response to chemotherapy.

Results

153 patients with SCLC were included for analysis with a median age of 66 years (34-90), 72.8% men and the median overall survival of 9.37 months. Of these, 75% patients had extensive stage disease and 35% were limited stage. No statistically significant difference was found in baseline clinical characteristics between R and NR. A multivariate Cox regression analysis indicated that RRS was significantly associated with OS in the training set [HR: 1.53; 95% confidence interval (CI), 1.1-2.2; P=0.021; C-index=0.72) and validation set [HR: 1.4; 95% CI, 1.1-1.82; P=0.0127; C-index=0.69). Chemotherapy response was achieved in 71 (66%) patients; labeled responders (R) and the rest 36 (34%) were labeled non-responders (NR). An LDA classifier trained with prognostic features was able to predict response to chemotherapy with an AUC of 0.76±0.03 within the training set and a corresponding AUC of 0.72 within the validation set. A multivariate Cox regression analysis with radiomic features and clinical biomarkers identified the RRS and cancer stage (limited or extensive) as two risk factors in OS for patients in the training set (RRS: HR, 2.1, 95% CI: 1.53, 2.85, P=0.0076; clinical stage: HR, 1.66, 95% CI: 1.01, 2.7, P=0.048; race: HR, 0.37, 95% CI: 0.12, 1.1, P=0.071; and age: HR, 1.04, 95% CI: 0.99, 1.09, P=0.071; C-index=0.75) and corresponding validation set (RRS: HR, 1.9, 95% CI: 1.23, 2.2, P=0.0012; clinical stage: HR, 1.61, 95% CI: 1.2, 2.17, P=0.041; race: HR, 0.86, 95% CI: 0.52, 1.42, P=0.56; and age: HR, 1.01, 95% CI: 0.99, 1.03, P=0.22; C-index=0.71).

Further, the median OS was 9.37 months (range: 0.2-52 months). A univariable Cox regression analysis identified that OS was not significantly different for gender (male vs. female; HR: 0.78; 95% CI, 0.48-1.25; P=0.3; C-index=0.52) or race (HR: 0.86; 95% CI, 0.53-1.4; P=0.54; C-index=0.51) but was significantly different for clinical stage (LS vs. ES; HR: 1.4; 95% CI, 1.3-1.7; P=0.0002; C-index=0.58) (See, e.g., 802-806). More specifically, patients with brain metastasis demonstrated poorer survival but in our dataset this finding was not significantly associated with OS (HR: 0.52; 95% CI, 0.27-0.98; P=0.069; C-index=0.57).

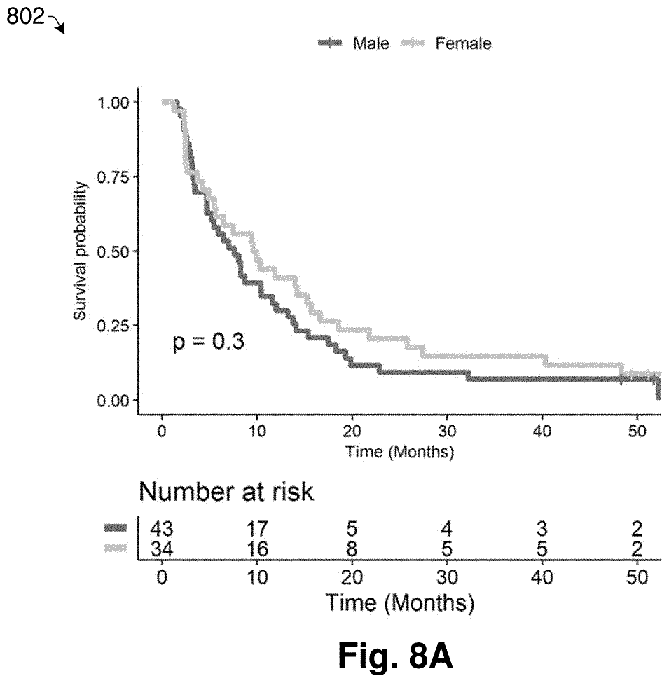

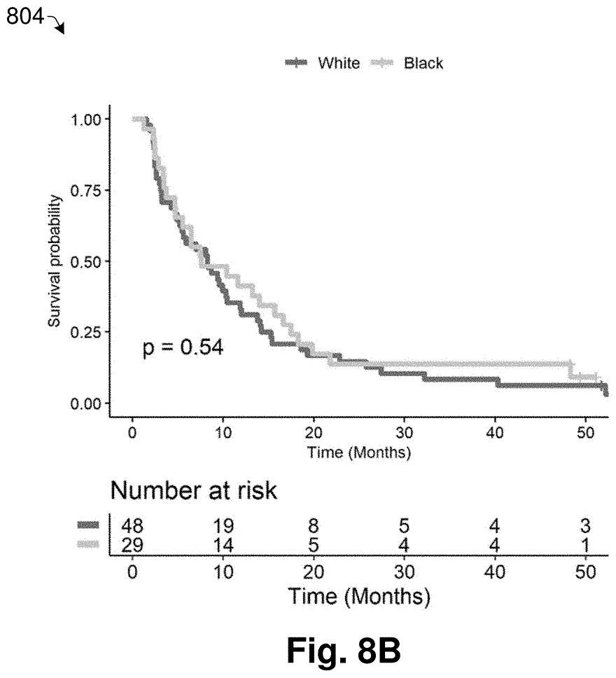

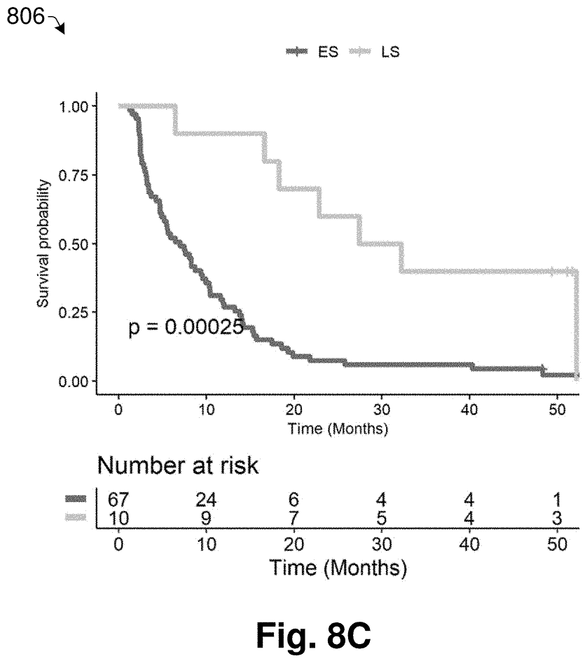

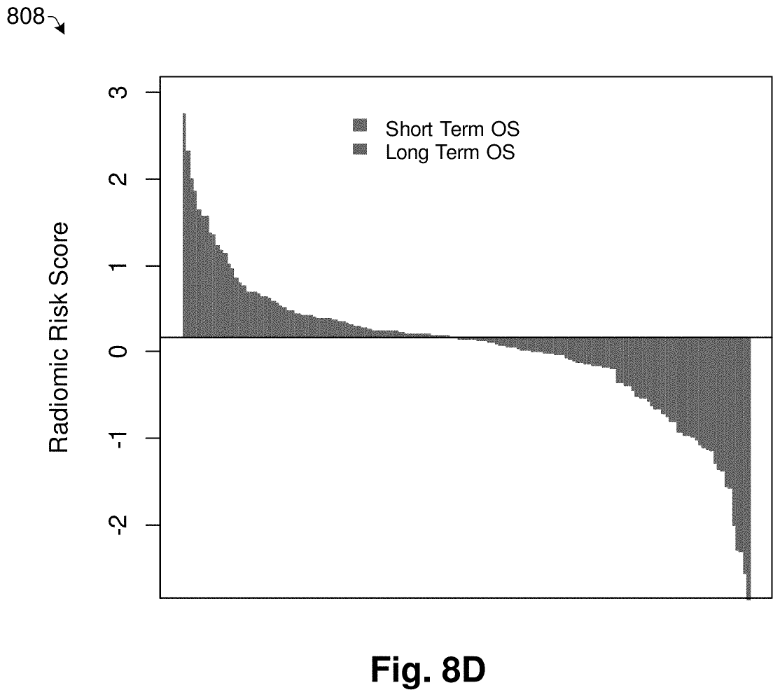

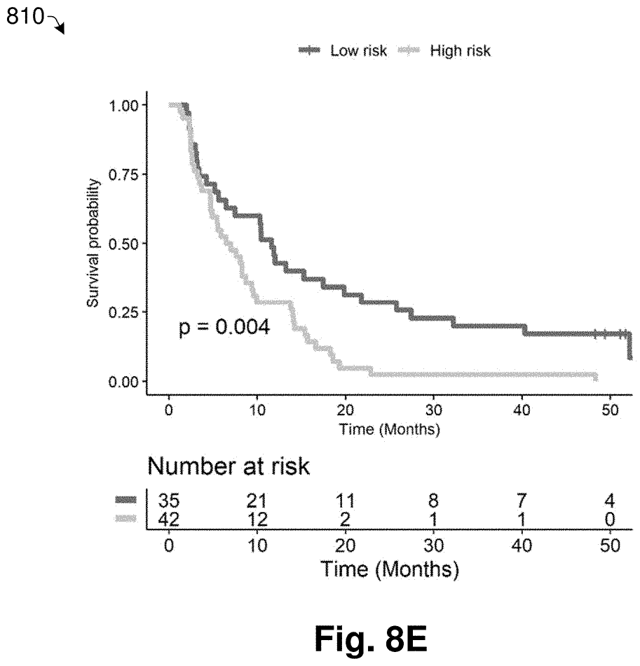

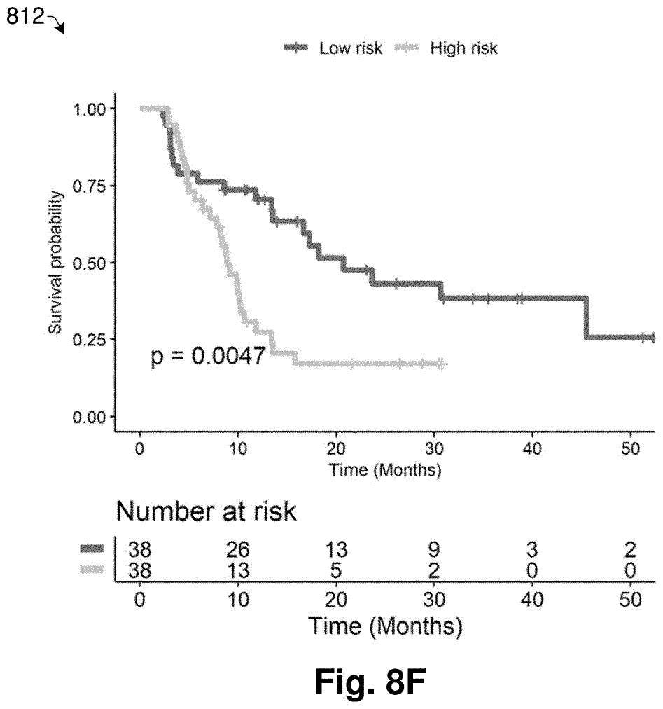

Moreover, the RRS was calculated as a linear combination of 6 selected features weighted by their respective coefficients. These features were identified as entropy of intratumoral Haralick feature, median of intratumoral Law texture feature, peritumoral laws texture feature, intratumoral low frequency Gabor feature and peritumoral high frequency Gabor feature. The optimum cut-off value (the median) for the RRS was found to be 0.17, and patients were stratified into high- and low-risk groups based on this value (See, e.g., 808). A significant association of the RSS with the OS was shown in the training set (P=0.004) and test set (P=0.0047). FIG. 8 illustrates various plots that are associated with Example Use Case 1.

More specifically, FIG. 8 illustrates first, second, and third Kaplan-Meier survival curves 802-806 for different clinical factors. The first Kaplan-Meier survival curve 802 is for gender (e.g., male vs. female) on the training set. The second Kaplan-Meier survival curve 804 is for race (e.g., white vs. black) on the training set. The third Kaplan-Meier survival curve 806 is for clinical stage (e.g., ES vs. LS) on the training set. Further, FIG. 8 illustrates a waterfall plot 808 of the length of OS based on RRS (higher risk score is associated with lower OS). Moreover, FIG. 8 illustrates a fourth Kaplan-Meier survival curve 810 according to the RRS for patients in the training dataset. In addition, FIG. 8 illustrates a fifth Kaplan-Meier survival curve 812 according to the RRS for patients in the validation dataset.

### CONCLUSION

Texture features extracted from within and around the lung tumor from pre-treatment CT images were both prognostic of OS and predictive of response to platinum-based chemotherapy in SCLC. Pre-treatment radiomic features may permit early assessment of benefit and expedite alternative treatment options especially in non-responders.

As demonstrated by the example embodiments, various embodiments can facilitate generating a prediction for a response to a treatment for SCLC based on radiomic features extracted from X-ray images (e.g., CT images). Being able to classify patients into either a responder group (RG) or a non-responder group (NRG) based on the radiomic features may allow a medical practitioner to easily and timely predict the patient's response to the SCLC treatment. Accordingly, the medical practitioner may be able to accurately guide the SCLC treatment of the patient to achieve better treatment results (e.g., to expedite alternative treatment options (e.g., adjuvant therapy, active surveillance, etc.) for the patient, if needed). Embodiments thus provide a measurable improvement over existing methods, systems, apparatus, or other devices in predicting response to the SCLC treatment.

In various example embodiments, method(s) discussed herein can be implemented as computer executable instructions. Thus, in various embodiments, a computer-readable storage device can store computer executable instructions that, when executed by a machine (e.g., computer, processor), cause the machine to perform methods or operations described or claimed herein including operation(s) described in connection with methods or operations 100, 200, 300, 400, 500, or 600, or any other methods or operations described herein. While executable instructions associated with the listed methods are described as being stored on a computer-readable storage device, it is to be appreciated that executable instructions associated with other example methods or operations described or claimed herein can also be stored on a computer-readable storage device. In different embodiments, the example methods or operations described herein can be triggered in different ways. In one embodiment, a method or operation can be triggered manually by a user. In another example, a method or operation can be triggered automatically.

Embodiments discussed herein related to generating a prediction for a response to a treatment for SCLC are based on radiomic features that are not perceivable by the human eye, and their computation cannot be practically performed in the human mind. A machine learning classifier as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

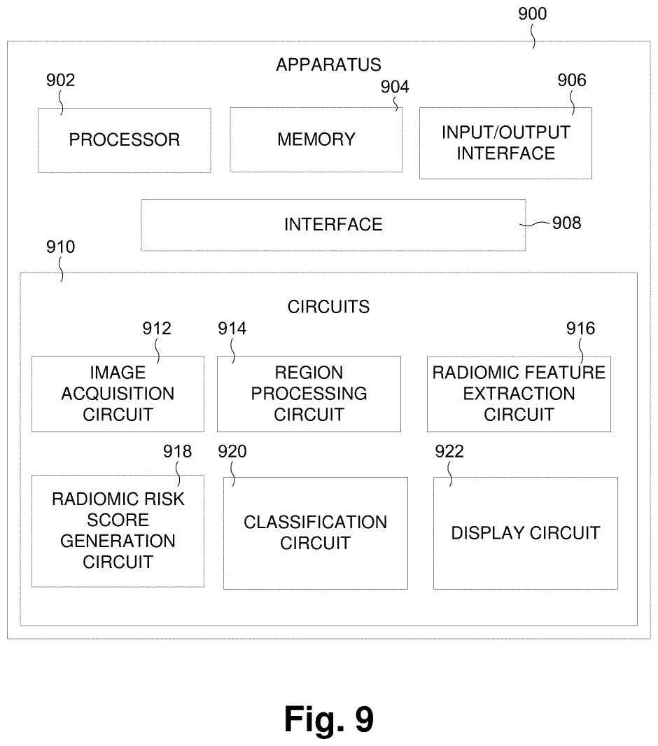

FIG. 9 illustrates some embodiments of an apparatus that can facilitate the methods described herein. For example, FIG. 9 illustrates some embodiments of an apparatus 900 that can facilitate predicting a new patient's response to a SCLC treatment based on radiomic features extracted from an X-ray image of the new patient (e.g., a patient that is receiving or is to receive treatment for SCLC), according to various embodiments discussed herein.

Apparatus 900 may be configured to perform various techniques, operations, or methods discussed herein, for example, training a machine learning classifier (e.g., linear discriminant analysis, quadratic discriminant analysis classifier, support vector machine, etc.) based on a training dataset to predict a patient's response to the SCLC treatment, and/or employing the trained machine learning classifier to generate a classification of the new patient into either a responder or non-responder group. In one embodiment, apparatus 900 includes a processor 902, and a memory 904. Processor 902 may, in various embodiments, include circuitry such as, but not limited to, one or more single-core or multi-core processors. Processor 902 may include any combination of general-purpose processors and dedicated processors (e.g., graphics processors, application processors, etc.). The processor(s) can be coupled with and/or can comprise memory (e.g., memory 904) or storage and can be configured to execute instructions stored in the memory 904 or storage to enable various apparatus, applications, or operating systems to perform operations and/or methods discussed herein.

Memory 904 is configured to store an X-ray image (or a plurality of X-ray images) of the new patient, where the X-ray image comprises a pulmonary lesion that is indicative of SCLC. In some embodiments, memory 904 can also store a training dataset of X-ray images for training the machine learning classifier (e.g., linear discriminant analysis classifier, etc.), and/or a validation dataset of X-ray images. Memory 904 can be further configured to store one or more clinical features (e.g., cancer stage) or other data associated with the new patient.

Apparatus 900 also includes an input/output (I/O) interface 906; a set of circuits 910; and an interface 908 that connects the processor 902, the memory 904, the I/O interface 906, and the set of circuits 910. I/O interface 906 may be configured to transfer data between memory 904, processor 902, circuits 910, and external devices, for example, a medical imaging device such as a CT system.

The set of circuits 910 includes an image acquisition circuit 912, a region processing circuit 914, a radiomic feature extraction circuit 916, a radiomic risk score generation circuit, a classification circuit 920, and a display circuit 922.

The image acquisition circuit 912 is configured to access the X-ray image(s) of the new patient, according to embodiments and examples described. The image acquisition circuit 912 may also be configured to access the X-ray image(s) of the validation dataset and/or training dataset, according to embodiments and examples described. Accessing the X-ray image(s) may include accessing the X-ray image(s) stored in memory 904. In another embodiment accessing the X-ray image(s) may include acquiring electronic data, reading from a computer file, receiving a computer file, reading from a computer memory, or other computerized activity not practically performed in the human mind.

Region processing circuit 914 is configured to define an intratumoral region and a peritumoral region of a pulmonary lesion on the X-ray image(s), according to embodiments and examples described.

Radiomic feature extraction circuit 916 is configured to extract a plurality of radiomic features from the X-ray image(s), according to embodiments and examples described.

Radiomic risk score (RRS) generation circuit 918 is configured to generate a RRS for the new patient based on the X-ray images(s) of the new patient, according to embodiments and examples described. The RRS generation circuit 918 may also be configured to generate RRSs for the X-ray image(s) of the validation dataset and/or the X-ray images of the training dataset, according to embodiments and examples described.

Classification circuit 920 is configured to classify the new patient into either a responder group (RG) or a non-responder group (NRG) based, at least in part, on the RRS of the new patient, according to embodiments and examples described. The classification circuit 920 may also be configured to classify past SCLC patients (e.g., of the validation/training dataset) into either the RG or NRG, according to embodiments and examples described.

Display circuit 922 is configured to display the classification of the new patient (e.g., whether the new patient is in the RG or the NRG), according to embodiments and examples described. The display circuit 922 may also be configured to display the classification of past SCLC patients (e.g., of the validation/training dataset), according to embodiments and examples described.

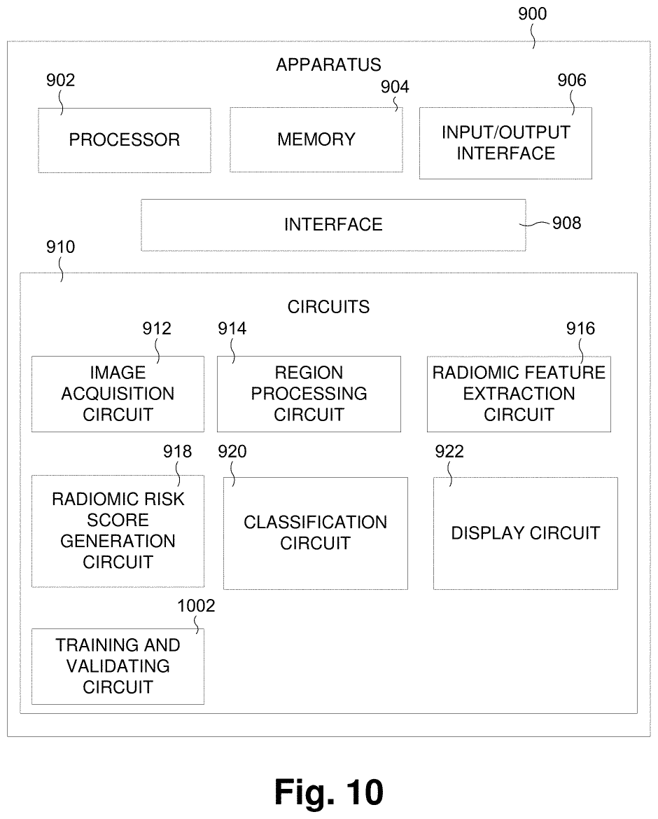

FIG. 10 illustrates some other embodiments of the apparatus 900 of FIG. 9. As shown in FIG. 10, in some embodiments, the set of circuits 910 further includes a training and validating circuit 1002. The training and validating circuit 1002 is configured to train the classification circuit 920 on a training dataset (e.g., a training cohort); and optionally validate the classification circuit 920 on a validation dataset, according to various embodiments described herein (e.g., train and validate the machine learning classifier).

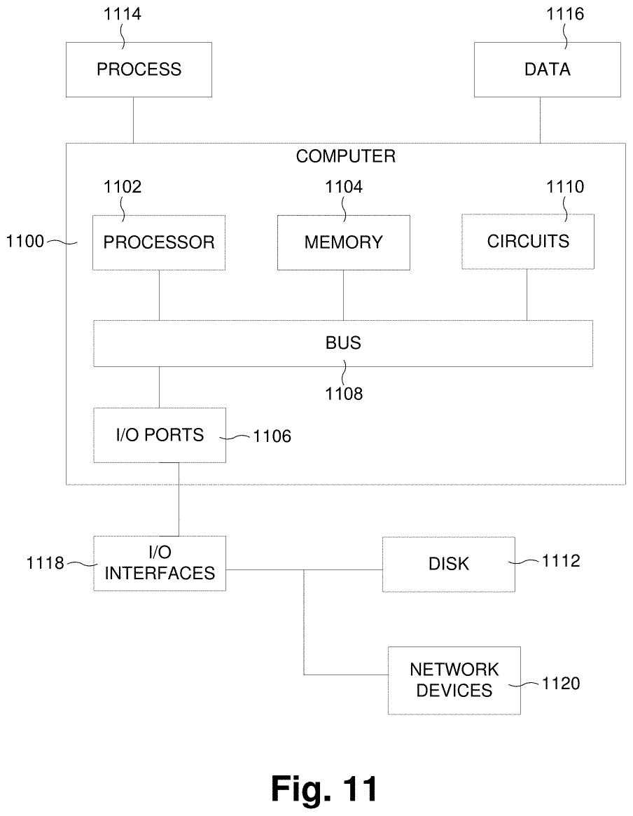

FIG. 11 illustrates some embodiments of a computer in which methods described herein can operate and in which example methods, apparatus, circuits, operations, or logics may be implemented. In different examples, computer 1100 may be part of a SCLC treatment prediction system or apparatus, an MRI system, a CT system, or may be operably connectable to a SCLC treatment prediction system or apparatus, an MRI system, a CT system.

Computer 1100 includes a processor 1102, a memory 1104, and input/output (I/O) ports 1106 operably connected by a bus 1108. In one example, computer 1100 may include a set of logics or circuits 1110 that perform operations for or a method of generating a prediction for a response to a treatment for SCLC and/or a prediction for overall survival (OS) of a patient with SCLC, according to embodiments and examples described.

Thus, the set of circuits 1110, whether implemented in computer 1100 as hardware, firmware, software, and/or a combination thereof may provide means (e.g., hardware, firmware, circuits) for generating a prediction for a response to a treatment for SCLC and/or a prediction for overall survival (OS) of a patient with SCLC, according to embodiments and examples described. In different examples, the set of circuits 1110 may be permanently and/or removably attached to computer 1100.

Processor 1102 can be a variety of various processors including dual microprocessor and other multi-processor architectures. Processor 1102 may be configured to perform steps of methods claimed and described herein. Memory 1104 can include volatile memory and/or non-volatile memory. A disk 1112 may be operably connected to computer 1100 via, for example, an input/output interface 1118 (e.g., card, device) and an input/output port 1106. Disk 1112 may include, but is not limited to, devices like a magnetic disk drive, a tape drive, a Zip drive, a flash memory card, or a memory stick. Furthermore, disk 1112 may include optical drives like a CD-ROM or a digital video ROM drive (DVD ROM). Memory 1104 can store processes 1114 or data 1116, for example. Data 1116 may, in one embodiment, include digitized radiological images, including X-ray images (e.g. CT images) having pulmonary lesions that are indicative of SCLC, according to embodiments and examples described. Disk 1112 or memory 1104 can store an operating system that controls and allocates resources of computer 1100.

Bus 1108 can be a single internal bus interconnect architecture or other bus or mesh architectures. While a single bus is illustrated, it is to be appreciated that computer 1100 may communicate with various devices, circuits, logics, and peripherals using other buses that are not illustrated (e.g., PCIE, SATA, Infiniband, IEEE 1394, USB, Ethernet).

Computer 1100 may interact with input/output devices via I/O interfaces 1118 and the input/output ports 1106. Input/output devices can include, but are not limited to, MRI systems, CT systems, a keyboard, a microphone, a pointing and selection device, cameras, video cards, displays, disk 1112, network devices 1120, or other devices. The input/output ports 1106 can include but are not limited to, serial ports, parallel ports, or USB ports.

Computer 1100 may operate in a network environment and thus may be connected to network devices 1120 via I/O interfaces 1118 or I/O ports 1106. Through the network devices 1120, computer 1100 may interact with a network. Through the network, computer 1100 may be logically connected to remote computers. The networks with which computer 1100 may interact include, but are not limited to, a local area network (LAN), a wide area network (WAN), or other networks, including the cloud.

Examples herein can include subject matter such as an apparatus, an MRI system, a CT system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system for generating a prediction for a response to a treatment for SCLC, according to embodiments and examples described.

In some embodiments, the present application provides a method for predicting a response to treatment of small cell lung cancer (SCLC). The method comprises accessing an X-ray image of a patient that is receiving or is to receive treatment for SCLC, wherein the X-ray image comprises a pulmonary lesion, and wherein the X-ray image is from a computed tomography (CT) scan of the patient. An intratumoral region of the pulmonary lesion is defined. A peritumoral region of the pulmonary lesion is defined. A first plurality of radiomic features are extracted from the X-ray image, wherein each of the radiomic features of the first plurality of radiomic features relates to at least one of the intratumoral region of the pulmonary lesion and the peritumoral region of the pulmonary lesion. A radiomic risk score (RRS) is generated for the patient based on the first plurality of radiomic features, wherein the RRS is prognostic of overall survival (OS) of the patient. The RRS is provided to a machine learning classifier that is trained to predict a response of the patient to a SCLC chemotherapy treatment based, at least in part, on the RRS. From the machine learning classifier, a classification of the patient into either a responder group (RG) or a non-responder group (NRG) is received, where the NRG indicates the patient will not respond to the SCLC chemotherapy treatment and the RG indicates that the patient will respond to the SCLC chemotherapy treatment. The classification is displayed.

In some embodiments, extracting the first plurality of radiomic features from the X-ray image comprises: extracting a second plurality of radiomic features from the X-ray image, wherein each of the radiomic features of the second plurality of radiomic features relates to at least one of the intratumoral region of the pulmonary lesion and the peritumoral region of the pulmonary lesion; and selecting a subset of radiomic features of the second plurality of radiomic features, wherein the subset of radiomic features of the second plurality of radiomic features are more relevant to predicting OS of patients with SCLC than the other radiomic features of the second plurality of radiomic features for a predefined feature selection process, and wherein the subset of radiomic features defines the first plurality of radiomic features.

In some embodiments, each of the radiomic features of the first plurality of radiomic features are either a shape-based feature of the intratumoral region of the pulmonary lesion, a texture-based feature of the intratumoral region of the pulmonary lesion, a shape-based feature of the peritumoral region of the pulmonary lesion, or a texture-based feature of the peritumoral region of the pulmonary lesion.

In some embodiments, the first plurality of radiomic features comprises a Haralick feature, a Laws feature, and a Gabor feature.

In some embodiments, the first plurality of radiomic features comprises a Haralick entropy feature of the intratumoral region, a Laws texture feature of the intratumoral region, a Laws texture feature of the peritumoral region, a low frequency Gabor feature of the intratumoral region, and a high frequency Gabor feature of the peritumoral region.

In some embodiments, the SCLC chemotherapy treatment is a platinum-based chemotherapy treatment.

In some embodiments, the X-ray image of the patient is a pre-treatment X-ray image of the patient.

In some embodiments, generating the RRS comprises: assigning a value to each of the radiomic features of the first plurality of radiomic features; and combining the value of each of the radiomic features of the first plurality of radiomic features to generate the RRS.

In some embodiments, the RRS is generated using a least absolute shrinkage and selection operator (LASSO) technique.

In some embodiments, defining the intratumoral region of the pulmonary lesion comprises: defining an outer boundary of the pulmonary lesion; and defining an area within the outer boundary of the pulmonary lesion as the intratumoral region of the pulmonary lesion.

In some embodiments, defining the peritumoral region of the pulmonary lesion comprises: enlarging the outer boundary of the pulmonary lesion by about 15 millimeters to define an outer boundary of the peritumoral region of the pulmonary lesion; and defining an area between the outer boundary of the pulmonary lesion and the outer boundary of the peritumoral region as the peritumoral region of the pulmonary lesion.

In some embodiments, the method further comprises providing the RRS and another, different prognostic feature of OS of the patient to the machine learning classifier, wherein the machine learning classifier is trained to predict the response of the patient to the SCLC chemotherapy treatment based on a combination of the RRS and the another, different prognostic feature of OS of the patient.

In some embodiments, the another, different prognostic feature of OS is a stage of the patient's SCLC.

In some embodiments, the stage of the patient's SCLC is either extensive stage or limited stage.

In some embodiments, the present application provides a non-transitory computer-readable storage device storing computer-executable instructions that when executed cause a processor to perform operations. The operations comprise: accessing an X-ray image associated with a patient, wherein the X-ray image comprises a portion of a pulmonary lesion that is indicative of small cell lung cancer (SCLC), and wherein the X-ray image is from a computed tomography (CT) scan of the patient; defining a perimeter of the portion of the pulmonary lesion; defining an area within the perimeter of the portion of the pulmonary lesion as an intratumoral region of the portion of the pulmonary lesion; enlarging the perimeter of the portion of the pulmonary lesion to define an enlarged perimeter of the portion of the pulmonary lesion; defining an area between the perimeter of the portion of the pulmonary lesion and the enlarged perimeter of the portion of the pulmonary lesion as a peritumoral region of the portion of the pulmonary lesion; extracting a first set of radiomic features from the X-ray image, wherein each of the radiomic features of the first set of radiomic features relates to at least one of the intratumoral region of the portion of the pulmonary lesion and the peritumoral region of the portion of the pulmonary lesion; generating a radiomic risk score (RRS) for the patient based on the first set of radiomic features, wherein the RRS is prognostic of overall survival (OS) of the patient; classifying the patient into either a short-term OS group or a long-term OS group by comparing the RRS of the patient to a threshold RRS value; and displaying the classification of the patient.

In some embodiments, classifying the patient into either the short-term OS group or the long-term OS group by comparing the RRS for the patient to the threshold RRS value comprises: classifying the patient into the short-term OS group if the RRS for the patient is less than the threshold RRS value; and classifying the patient into the long-term OS group if the RRS for the patient is greater than or equal to the threshold RRS value.

In some embodiments, extracting the first set of radiomic features from the X-ray image comprises: extracting a second set of radiomic features from the X-ray image, wherein each of the radiomic features of the second set of radiomic features relates to at least one of the intratumoral region of the portion of the pulmonary lesion and the peritumoral region of the portion of the pulmonary lesion; and selecting a subset of radiomic features of the second set of radiomic features, wherein the subset of radiomic features of the second set of radiomic features are more relevant to predicting OS of patients with SCLC than the other radiomic features of the second set of radiomic features, and wherein the subset of radiomic features defines the first set of radiomic features.

In some embodiments, the operation further comprise: extracting a plurality of sets of radiomic features from a plurality of X-ray images, wherein the plurality of X-ray images are associated with the patient, wherein each of the X-ray images of the plurality of X-ray images comprises a corresponding portion of the pulmonary lesion, wherein the X-ray image is a first X-ray image of the plurality of X-ray images, wherein the first set of radiomic features is one of the sets of the plurality of sets of radiomic features; assigning a value to each radiomic feature of the sets of radiomic features based on the plurality of the X-ray images; categorizing the radiomic features of each set of radiomic features into radiomic feature types; combining the values of the radiomic features that have the same radiomic feature type together to generate a plurality of combined values, wherein each of the plurality of combined values corresponds to one of the radiomic feature types; and generating the RRS for the patient based on the combined values.

In some embodiments, the present application provides a non-transitory computer-readable storage device storing computer-executable instructions that when executed cause a processor to perform operations. The operations comprising: accessing a training dataset of X-ray images, wherein the training dataset of X-ray images comprises a plurality of X-ray images, wherein each X-ray image demonstrates a pulmonary lesion that is indicative of small cell lung cancer (SCLC), wherein each X-ray image of the plurality of X-ray images is associated with a past SCLC patient, and wherein each X-ray image is from a computed tomography (CT) scan of a corresponding past SCLC patient; defining an intratumoral region for each of the pulmonary lesions; defining a peritumoral region for each of the pulmonary lesions; extracting a plurality of groups of radiomic features from the plurality of X-ray images, wherein each group of the plurality of groups of radiomic features is associated with a corresponding X-ray image of the plurality of X-ray images, and wherein, for each group of radiomic features, each radiomic feature relates to at least one of an attribute of a corresponding intratumoral region and an attribute of a corresponding peritumoral region; refining the plurality of groups of radiomic features to a plurality of subgroups of radiomic features, respectively, such that each subgroup of radiomic features of the plurality of subgroups of radiomic features is associated with a corresponding X-ray image of the plurality of X-ray images, wherein the radiomic features of the subgroups of radiomic features are more relevant to predicting overall survival (OS) of the past SCLC patients than the other radiomic features of the plurality of groups of radiomic features; generating radiomic risk scores (RRSs) for the past SCLC patients, respectively, wherein each RRS of the RRSs is generated based on a corresponding subgroup of radiomic features, and wherein each RRS is prognostic of OS of a corresponding past SCLC patient; and training a linear discriminant analysis (LDA) classifier based, at least in part, on the RRSs, wherein the LDA classifier is trained to predict a response to a platinum-based chemotherapy treatment for a new SCLC patient.

In some embodiments, training the LDA is based, at least in part, on the RRSs and SCLC stages of the past SCLC patients.

Examples herein can include subject matter such as an apparatus, including an NSCLC immunotherapy response prediction apparatus or system, a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system for predicting NSCLC immunotherapy response, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method of predicting a response to treatment of small cell lung cancer (SCLC), comprising:
accessing an X-ray image of a patient that is receiving or is to receive treatment for SCLC, wherein the X-ray image comprises a pulmonary lesion, and wherein the X-ray image is from a computed tomography (CT) scan of the patient;
defining an intratumoral region of the pulmonary lesion;
defining a peritumoral region of the pulmonary lesion;
extracting a first plurality of radiomic features from the X-ray image, wherein each of the radiomic features of the first plurality of radiomic features relates to at least one of the intratumoral region of the pulmonary lesion and the peritumoral region of the pulmonary lesion;
generating a radiomic risk score (RRS) for the patient based on the first plurality of radiomic features, wherein the RRS is prognostic of overall survival (OS) of the patient;
providing the RRS to a machine learning classifier that is trained to predict a response of the patient to a SCLC chemotherapy treatment based, at least in part, on the RRS;
receiving, from the machine learning classifier, a classification of the patient into either a responder group (RG) or a non-responder group (NRG), where the NRG indicates the patient will not respond to the SCLC chemotherapy treatment and the RG indicates that the patient will respond to the SCLC chemotherapy treatment; and
displaying the classification.

2. The method of claim 1, wherein extracting the first plurality of radiomic features from the X-ray image comprises:
extracting a second plurality of radiomic features from the X-ray image, wherein each of the radiomic features of the second plurality of radiomic features relates to at least one of the intratumoral region of the pulmonary lesion and the peritumoral region of the pulmonary lesion; and
selecting a subset of radiomic features of the second plurality of radiomic features, wherein the subset of radiomic features of the second plurality of radiomic features are more relevant to predicting OS of patients with SCLC than the other radiomic features of the second plurality of radiomic features for a predefined feature selection process, and wherein the subset of radiomic features defines the first plurality of radiomic features.

3. The method of claim 1, wherein:
each of the radiomic features of the first plurality of radiomic features are either a shape-based feature of the intratumoral region of the pulmonary lesion, a texture-based feature of the intratumoral region of the pulmonary lesion, a shape-based feature of the peritumoral region of the pulmonary lesion, or a texture-based feature of the peritumoral region of the pulmonary lesion.

4. The method of claim 3, wherein:
the first plurality of radiomic features comprises a Haralick feature, a Laws feature, and a Gabor feature.

5. The method of claim 4, wherein:
the first plurality of radiomic features comprises a Haralick entropy feature of the intratumoral region, a Laws texture feature of the intratumoral region, a Laws texture feature of the peritumoral region, a low frequency Gabor feature of the intratumoral region, and a high frequency Gabor feature of the peritumoral region.

6. The method of claim 1, wherein the SCLC chemotherapy treatment is a platinum-based chemotherapy treatment.

7. The method of claim 6, wherein the X-ray image of the patient is a pre-treatment X-ray image of the patient.

8. The method of claim 1, wherein generating the RRS comprises:
assigning a value to each of the radiomic features of the first plurality of radiomic features; and
combining the value of each of the radiomic features of the first plurality of radiomic features to generate the RRS.

9. The method of claim 8, wherein the RRS is generated using a least absolute shrinkage and selection operator (LASSO) technique.

10. The method of claim 1, wherein defining the intratumoral region of the pulmonary lesion comprises:
defining an outer boundary of the pulmonary lesion; and
defining an area within the outer boundary of the pulmonary lesion as the intratumoral region of the pulmonary lesion.

11. The method of claim 10, wherein defining the peritumoral region of the pulmonary lesion comprises:
enlarging the outer boundary of the pulmonary lesion by about 15 millimeters to define an outer boundary of the peritumoral region of the pulmonary lesion; and
defining an area between the outer boundary of the pulmonary lesion and the outer boundary of the peritumoral region as the peritumoral region of the pulmonary lesion.

12. The method of claim 1, further comprising:
providing the RRS and another, different prognostic feature of OS of the patient to the machine learning classifier, wherein the machine learning classifier is trained to predict the response of the patient to the SCLC chemotherapy treatment based on a combination of the RRS and the another, different prognostic feature of OS of the patient.

13. The method of claim 12, wherein the another, different prognostic feature of OS is a stage of the patient's SCLC.

14. The method of claim 13, wherein the stage of the patient's SCLC is either extensive stage or limited stage.

15. A non-transitory computer-readable storage device storing computer-executable instructions that when executed cause a processor to perform operations, the operations comprising:
accessing an X-ray image associated with a patient, wherein the X-ray image comprises a portion of a pulmonary lesion that is indicative of small cell lung cancer (SCLC), and wherein the X-ray image is from a computed tomography (CT) scan of the patient;
defining a perimeter of the portion of the pulmonary lesion;
defining an area within the perimeter of the portion of the pulmonary lesion as an intratumoral region of the portion of the pulmonary lesion;
enlarging the perimeter of the portion of the pulmonary lesion to define an enlarged perimeter of the portion of the pulmonary lesion;
defining an area between the perimeter of the portion of the pulmonary lesion and the enlarged perimeter of the portion of the pulmonary lesion as a peritumoral region of the portion of the pulmonary lesion;
extracting a first set of radiomic features from the X-ray image, wherein each of the radiomic features of the first set of radiomic features relates to at least one of the intratumoral region of the portion of the pulmonary lesion and the peritumoral region of the portion of the pulmonary lesion;
generating a radiomic risk score (RRS) for the patient based on the first set of radiomic features, wherein the RRS is prognostic of overall survival (OS) of the patient;
classifying the patient into either a short-term OS group or a long-term OS group by comparing the RRS of the patient to a threshold RRS value; and
displaying the classification of the patient.

16. The non-transitory computer-readable storage device of claim 15, wherein classifying the patient into either the short-term OS group or the long-term OS group by comparing the RRS for the patient to the threshold RRS value comprises:
classifying the patient into the short-term OS group if the RRS for the patient is less than the threshold RRS value; and
classifying the patient into the long-term OS group if the RRS for the patient is greater than or equal to the threshold RRS value.

17. The non-transitory computer-readable storage device of claim 15, wherein extracting the first set of radiomic features from the X-ray image comprises:
extracting a second set of radiomic features from the X-ray image, wherein each of the radiomic features of the second set of radiomic features relates to at least one of the intratumoral region of the portion of the pulmonary lesion and the peritumoral region of the portion of the pulmonary lesion; and
selecting a subset of radiomic features of the second set of radiomic features, wherein the subset of radiomic features of the second set of radiomic features are more relevant to predicting OS of patients with SCLC than the other radiomic features of the second set of radiomic features, and wherein the subset of radiomic features defines the first set of radiomic features.

18. The non-transitory computer-readable storage device of claim 15, the operations further comprising:
extracting a plurality of sets of radiomic features from a plurality of X-ray images, wherein the plurality of X-ray images are associated with the patient, wherein each of the X-ray images of the plurality of X-ray images comprises a corresponding portion of the pulmonary lesion, wherein the X-ray image is a first X-ray image of the plurality of X-ray images, wherein the first set of radiomic features is one of the sets of the plurality of sets of radiomic features;
assigning a value to each radiomic feature of the sets of radiomic features based on the plurality of the X-ray images;
categorizing the radiomic features of each set of radiomic features into radiomic feature types;
combining the values of the radiomic features that have the same radiomic feature type together to generate a plurality of combined values, wherein each of the plurality of combined values corresponds to one of the radiomic feature types; and
generating the RRS for the patient based on the combined values.

19. A non-transitory computer-readable storage device storing computer-executable instructions that when executed cause a processor to perform operations, the operations comprising:
accessing a training dataset of X-ray images, wherein the training dataset of X-ray images comprises a plurality of X-ray images, wherein each X-ray image demonstrates a pulmonary lesion that is indicative of small cell lung cancer (SCLC), wherein each X-ray image of the plurality of X-ray images is associated with a past SCLC patient, and wherein each X-ray image is from a computed tomography (CT) scan of a corresponding past SCLC patient;
defining an intratumoral region for each of the pulmonary lesions;
defining a peritumoral region for each of the pulmonary lesions;
extracting a plurality of groups of radiomic features from the plurality of X-ray images, wherein each group of the plurality of groups of radiomic features is associated with a corresponding X-ray image of the plurality of X-ray images, and wherein, for each group of radiomic features, each radiomic feature relates to at least one of an attribute of a corresponding intratumoral region and an attribute of a corresponding peritumoral region;
refining the plurality of groups of radiomic features to a plurality of subgroups of radiomic features, respectively, such that each subgroup of radiomic features of the plurality of subgroups of radiomic features is associated with a corresponding X-ray image of the plurality of X-ray images, wherein the radiomic features of the subgroups of radiomic features are more relevant to predicting overall survival (OS) of the past SCLC patients than the other radiomic features of the plurality of groups of radiomic features;
generating radiomic risk scores (RRSs) for the past SCLC patients, respectively, wherein each RRS of the RRSs is generated based on a corresponding subgroup of radiomic features, and wherein each RRS is prognostic of OS of a corresponding past SCLC patient; and
training a linear discriminant analysis (LDA) classifier based, at least in part, on the RRSs, wherein the LDA classifier is trained to predict a response to a platinum-based chemotherapy treatment for a new SCLC patient.

20. The non-transitory computer-readable storage device of claim 19, wherein training the LDA is based, at least in part, on the RRSs and SCLC stages of the past SCLC patients.
